(19) United States
(12) Patent Application Publication
(10) Pub. No.: US 2022/0323695 A1
(43) Pub. Date: Oct. 13, 2022

(54) DRUG DELIVERY SYSTEMS AND METHODS

(71) Applicant: JANSSEN PHARMACEUTICALS, INC., Titusville, NJ (US)

(72) Inventors: Francesco N. ALBERTINI, Pleasanton, CA (US); Gregory J. BAKOS, Mason, OH (US); Anthony R. DIUBALDI, Jackson, NJ (US); Jason L. HARRIS, Lebanon, OH (US); Emma Louise HUBERT, San Jose, CA (US); Michael HUTCHINSON, King of Prussia, PA (US); Monica A. KAPIL, San Jose, CA (US); Peter KRULEVITCH, Pleasanton, CA (US); Chaley John LARSON, Pottstown, PA (US); Wouter Jacques Noel LEQUIEU, Lievegem (BE); Frederick E. SHELTON, IV, Hillsboro, OH (US); Michael J. VENDELY, Lebanon, OH (US); Steven M. VESOLE, San Bruno, CA (US)

(21) Appl. No.: 17/762,802

(22) PCT Filed: Sep. 24, 2020
(86) PCT No.: PCT/IB2020/058956

§ 371 Date: Mar. 23, 2022

Related U.S. Application Data
(60) Provisional application No. 62/905,437, filed on Sep. 25, 2019.
(60) Provisional application No. 62/905,438, filed on Sep. 25, 2019.
(60) Provisional application No. 63/020,865, filed on May 6, 2020.

(51) Int. Cl. A61M 5/50; G16H 20/17

(52) U.S. Cl. CPC A61M 5/5086; G16H 20/17; A61M 2205/3324; A61M 2205/3368; A61M 2205/276

(57) ABSTRACT

Drug delivery systems and methods are provided for monitoring and/or tracking exposure of a drug to one or more conditions that can affect performance of the drug.

### FIELD

The embodiments described herein relate to a device for administering and/or provision of a drug. The present disclosure further relates to a system in which the device can be used, and a method of administration, and a further method associated with the system.

### BACKGROUND

Pharmaceutical products (including large and small molecule pharmaceuticals, hereinafter “drugs”) are administered to patients in a variety of different ways for the treatment of specific medical indications. Regardless of the manner of the administration, care must be taken when administering drugs to avoid adverse effects on the patient. For example, care must be taken not to administer more than a safe amount of the drug to the patient. This requires consideration of the amount of dose given and the time frame over which the dose is delivered, sometimes in relation to previous doses, or doses of other drugs. Moreover, care must be taken not to inadvertently administer an incorrect drug to the patient, or drugs that have degraded due to their age or storage conditions. All of these considerations can be conveyed in guidance associated with the specific drugs or drug combinations. However, this guidance is not always followed correctly, for example due to mistakes, such as human error. This can lead to adverse effects on the patient or result in inappropriate drug administration, for example insufficient or excessive volume of drug being administered for the specific medical indication.

Further, before the drug is administered to the patient, the drug can be exposed to various conditions, e.g., geographic location, time, temperature, humidity, ultraviolet electromagnetic radiation. This exposure can occur at any point along the drug's supply chain (e.g., manufacturing, packaging, storage, and distribution) and/or between intermittent dosing. Such intermittent and cumulative exposure to environmental conditions can lead to adverse effects on the potency and stability of the drug, thereby decreasing efficacy and shelf-life. In fact, in some instances, certain exposure conditions can ultimately render the drug non-viable for use.

In relation to how a drug is administered to the patient, there are various dosage forms that can be used. For example, these dosage forms may include parenteral, inhalational, oral, ophthalmic, nasal, topical, and suppository forms of one or more drugs.

The dosage forms can be administered directly to the patient via a drug administration device. There are a number of different types of drug administration devices commonly available for delivery of the various dosage forms including: syringes, injection devices (e.g., autoinjectors, jet injectors, and infusion pumps), nasal spray devices, and inhalers.

It can be desirable to monitor compliance with the guidance that is associated with the drugs that are administered to a patient in various dosage forms. This can provide assurance that correct procedures are being followed and avoid the adoption of incorrect and potentially dangerous approaches. Further, this can also enable optimization of the administration of the drug to the patient.

### SUMMARY

Drug delivery systems, and methods are provided for monitoring the exposure of a drug to one or more exposure conditions.

In one exemplary embodiment, a drug delivery system is provided and includes a drug administration device, a first sensor, and second sensor. The drug administration device includes a drug holder having a drug disposed therein, and the drug administration device is configured to deliver the drug. The first sensor is associated with at least one of the drug administration device and a packaging unit for the drug administration device, and the first sensor is configured to monitor at least one exposure condition of the drug after being associated with the drug administration device. The second sensor is associated with the drug and is configured to monitor at least one exposure condition of the drug from an initial time before the drug is associated with the drug administration device to a second time in which the drug is associated with the drug administration device and the first sensor is activated.

The drug delivery system can vary in one or more ways. For example, the first sensor can be on the packaging unit and the packaging unit contains one or more drug administration devices. For another example, the at least one exposure condition can be at least one of geographic location, time, date, temperature, UV exposure, and humidity. For still another example, the drug administration device can be one of a blister pack, an autoinjector, an infusion pump, a nasal spray device, and an inhaler. For another example, the drug administration device can further include a drug dispensing mechanism that can be configured to deliver at least a portion of the drug upon actuation of a drug delivery actuator by a user. For yet another example, the drug delivery system can also include a communications interface configured to communicate with a processor. In at least some embodiments, the processor can be one of a processor remote from the drug administration device and a processor local to the drug administration device.

For another example, the initial time can be the time that the drug enters the supply chain. In at least some embodiments, the data collected by at least one of the first sensor and the second sensor can be configured to be communicated to the processor via the communications interface. In at least some embodiments, the processor can be configured to determine an expiration date for at least one of a batch of the drug is beyond its expiration date and the drug in the drug administration device. In at least some embodiments, the processor can be configured to generate a warning to a user in response to determining that at least one of a batch of the drug is beyond its expiration date and the drug in the drug administration device is beyond its expiration date, and/or the drug administration device can be configured to prevent drug delivery in the event that at least one of a batch of the drug and the drug in the drug administration device is beyond its expiration date.

For another example, the initial time can be a time that the drug is packaged.

For yet another example, the drug can include at least one of infliximab, golimumab, ustekinumab, daratumumab, guselkumab, epoetin alfa, risperidone, esketamine, ketamine, and paliperidone palmitate.

In another exemplary embodiment, a method is provided and in one exemplary embodiment includes monitoring, by a first sensor, at least one exposure condition of a drug after the drug is associated with a drug administration device, transmitting data representative of the at least one exposure condition to a communications interface in communication with the first sensor, receiving and transmitting, by the communications interface, the data to a processor that is in communication with the communications interface, and determining, by the processor, viability of the drug based on the received data characterizing the at least one exposure condition.

The method can vary in one or more ways. For example, the method can further include generating, by the processor, a warning to a user in response to determining that the drug is nonviable. For yet another example, the method can further include preventing, by the processor, drug delivery in response to determining that the drug is nonviable. For still another example, the method can further include adjusting, by the processor, a dosage of the drug based on the determined viability. For another example, the method can also include monitoring, by a second sensor, at least one exposure condition of the drug during a time interval that is at least prior to the drug being associated with the drug administration device, transmitting data representative of the at least one exposure condition over the time interval to the communications interface in communication with the second sensor, receiving and transmitting, by the communications interface, the data to the processor, and determining, by the processor, viability of the drug based on the received data characterizing the at least one exposure condition. In at least some embodiments, the time interval can include when the drug enters the supply chain, the method can further include generating, by the processor, a warning to a user in response to determining that the drug is nonviable, the method can further include preventing, by the processor, drug delivery in response to determining that the drug is nonviable, and/or the method can further include adjusting, by the processor, a dosage of the drug based on the determined viability. In at least some embodiments, the time interval can include when the drug is packaged.

For yet another example, the drug can include at least one of infliximab, golimumab, ustekinumab, daratumumab, guselkumab, epoetin alfa, risperidone, esketamine, ketamine, and paliperidone palmitate.

In another exemplary embodiment, a drug delivery system includes a housing containing one or more drug administration devices, a sensor, and a communications interface. Each drug administration device includes at least one drug holder having a drug disposed therein, and each drug administration device is configured to deliver the drug. The sensor is associated with at least one of the housing, the one or more drug administration devices, and the at least one drug holder. The sensor is configured to detect at least one exposure condition of the drug. The communications interface is configured to communicate the at least one exposure condition of the drug to a processor.

The drug delivery system can vary in one or more ways. For example, the housing can be a packaging unit for the one or more drug administration devices. For another example, at least one of the one or more drug administration devices can be one of a blister pack, an autoinjector, an infusion pump, a nasal spray device, and an inhaler.

For yet another example, at least one of the one or more drug administration devices can further include a drug dispensing mechanism configured to deliver the drug upon actuation of a drug delivery actuator by a user. For still another example, at least one of the one or more drug administration devices can further include a local processor that can be configured to at least one of adjust a dosage of the drug based on the at least one exposure condition of the drug and adjust a rate of delivery of the drug from the one or more drug administration devices based on the at least one exposure condition of the drug.

For another example, the processor can be one of a processor remote from the one or more drug administration devices and a processor local to the one or more drug administration devices. In at least some embodiments, the at least one exposure condition can be at least one of geographic location, time, date, temperature, UV exposure, and humidity, and, in at least some embodiments, the communications interface can be configured to communicate data representative of the exposure condition to the processor at one of a regular sampling rate, on demand, and continuously.

For another example, the sensor can be configured to sense at least one of an intensity of the at least one exposure condition and a duration of the at least one exposure condition.

For still another example, the drug delivery system can also include an indicator associated with at least one of the at least one drug holder and the one or more drug administration devices that can be configured to communicate a condition of the drug to a user. In at least some embodiments, the condition of the drug can be at least one of an expiration date of the drug, a viability state of the drug, a potency of the drug, and a recommended dosage of the drug.

For yet another example, the drug can include at least one of infliximab, golimumab, ustekinumab, daratumumab, guselkumab, epoetin alfa, risperidone, esketamine, ketamine, and paliperidone palmitate.

In another exemplary embodiment, a method includes detecting, by at least one sensor, at least one exposure condition of a drug, transmitting data representative of the at least one exposure condition to a communications interface in communication with the at least one sensor, receiving and transmitting, by the communications interface, the data to a processor that is in communication with the communications interface, and determining, by the processor, viability of the drug based on the received data characterizing the at least one exposure condition.

The method can vary in one or more ways. For example, the method can further include generating, by the processor, a warning to a user in response to determining that the drug is nonviable. For yet another example, the method can further include preventing, by the processor, drug delivery in response to determining that the drug is nonviable. For still another example, the method can further include adjusting, by the processor, a dosage of the drug based on the determined viability. For yet another example, the drug can include at least one of infliximab, golimumab, ustekinumab, daratumumab, guselkumab, epoetin alfa, risperidone, esketamine, ketamine, and paliperidone palmitate.

In another exemplary embodiment, a drug delivery system includes a drug administration device that includes a drug holder having a drug disposed therein, a drug status indicator associated with at least one of the drug administration device and the drug holder, and a reader associated with the drug administration device. The drug administration device is configured to deliver the drug. The drug status indicator is configured to indicate an extent of an exposure of the drug to an environmental condition. The reader is configured to detect the drug status indicator.

The drug delivery system can vary in one or more ways. For example, the environmental condition can be at least one of temperature, UV exposure, pH, and humidity.

For another example, the drug status indicator can be on a housing for at least one of the drug administration device and the drug holder. In at least some embodiments, the housing can be a packaging unit for one or more drug administration devices.

For yet another example, the drug administration device can be one of a blister pack, an autoinjector, an infusion pump, a nasal spray device, and an inhaler. For still another example, the drug administration device can further include a drug dispensing mechanism configured to deliver the drug upon actuation of a drug delivery actuator by a user. For another example, the drug status indicator can be configured to be responsive to at least one of an intensity of the environmental condition and a duration of the environmental condition.

For yet another example, the drug status indicator can be a degradable element that can be configured to degrade in response to at least one of a threshold exposure duration and a threshold exposure intensity to the environmental condition, and the reader can be configured to be in communication with a processor that can be configured to prompt a cue to a user when at least one of the threshold exposure duration and the threshold exposure intensity has been exceeded. In at least some embodiments, the cue can be at least one of an audible cue and a visual cue.

For another example, the drug status indicator can be configured to undergo a color change in response to at least one of a threshold exposure duration or a threshold exposure intensity to the environmental condition, and the reader can be configured to be in communication with a processor that can be configured to prompt a cue to a user when at least one of the threshold exposure duration and the threshold exposure intensity has been exceeded. For still another example, the drug status indicator can be formed of at least one electrochromic material. For another example, the drug status indicator can be formed of at least one of polylactic acid, polyglycolic acid, polycaprolactone, and polydioxanone. For yet another example, the drug status indicator can include a reactive agent that can be configured to interact with the drug so as to provide a visual change that is configured to be detected by the reader when the drug is below at least one of a threshold exposure duration and a threshold exposure intensity to the environmental condition. For still another example, the drug holder can include a first vial configured to have a lyophilized component of the drug disposed therein and a second vial configured to have a diluent disposed therein, the lyophilized component can be configured to be mixed with the diluent to reconstitute the drug prior to delivery of the drug, and the drug status indicator can be integrated with the drug holder and can be configured to indicate whether the lyophilized component and the diluent are each in a safe state for mixing. For another example, the drug holder can include a first vial configured to have a lyophilized component of the drug disposed therein and a second vial configured to have a diluent disposed therein, the lyophilized component can be configured to be mixed with the diluent to reconstitute the drug prior to delivery of the drug, and the drug status indicator can be configured to be releasably and replaceably attached to the drug administration device and can be configured to indicate whether the lyophilized component and the diluent are each in a safe state for mixing.

For yet another example, the drug can include at least one of infliximab, golimumab, ustekinumab, daratumumab, guselkumab, epoetin alfa, risperidone, esketamine, ketamine, and paliperidone palmitate.

In another exemplary embodiment, a drug delivery system includes a drug administration device including a housing and a drug holder having a drug disposed therein, and a label associated with at least one of the housing and the drug holder. The drug administration device is configured to deliver the drug. The label is configured to provide a visual indication to a user that the drug has exceeded a temperature threshold for the drug.

The drug delivery system can vary in one or more ways. For example, the drug can include at least one of infliximab, golimumab, ustekinumab, daratumumab, esketamine, ketamine, and guselkumab. For another example, the temperature threshold can include at least one of an absolute minimum temperature threshold, an absolute maximum temperature threshold, and a duration threshold below the absolute minimum temperature threshold or above the absolute maximum temperature threshold. For yet another example, the label can include at least one of a thermochromic material and an electrochemical material. For another example, the label can include a reactive agent that is configured to interact with the drug so as to trigger a visual change of at least a portion of the label when the drug has exceeded a temperature threshold. For still another example, the visual indication can be a color change of at least a portion of the label from a first color to a second color that is different than the first color.

In another exemplary embodiment, a method includes monitoring, by a drug status indicator associated with at least one of a drug administration device and a drug holder having a drug disposed therein, an extent of an exposure of the drug to at least one environmental condition, sensing, by a reader associated with the drug administration device, the drug status indicator to detect a response that is indicative of the extent of the exposure of the drug to the at least one environmental condition, transmitting data representative of the response of the drug status indicator to a processor in communication with the reader, and determining, by the processor, viability of the drug based on the received data characterizing the response of the drug status indicator.

The method can vary in one or more ways. For example, the drug can include at least one of infliximab, golimumab, ustekinumab, daratumumab, guselkumab, epoetin alfa, risperidone, esketamine, ketamine, and paliperidone palmitate.

### BRIEF DESCRIPTION OF DRAWINGS

The present invention is described by way of reference to the accompanying figures which are as follows:

FIG. 1 is a schematic view of a first type of drug administration device, namely an autoinjector;

FIG. 2 is a schematic view of a second type of drug administration device, namely an infusion pump;

FIG. 3 is a schematic view of a third type of drug administration device, namely an inhaler;

FIG. 4 is a schematic view of a fourth type of drug administration device, namely a nasal spray device;

FIG. 5A is a schematic view of a general drug administration device;

FIG. 5B is a schematic view of a universal drug administration device;

FIG. 6 is a schematic view of a housing for a dosage form;

FIG. 7 is a schematic view of one embodiment of a communication network system with which the drug administration devices and housing can operate;

FIG. 8 is a schematic view of one embodiment of a computer system with which the drug administration devices and housing can operate;

FIG. 9A is a schematic view of one embodiment of a drug administration device having a first sensor disposed thereon and a drug holder having a second sensor disposed thereon, showing the drug holder prior to being inserted into the drug administration device.

FIG. 9B is a schematic view of the drug administration device and the drug holder of FIG. 9A, showing the drug holder after being inserted into the drug administration device.

FIG. 10 is a schematic view of one embodiment of a drug holder having a sensor disposed thereon;

FIG. 10A is a schematic view of one embodiment of a drug administration device having the drug holder of FIG. 10 disposed therein;

FIG. 11 is schematic view of another embodiment of a drug holder having a sensor disposed thereon;

FIG. 12 is a graph illustrating an exemplary embodiment of sensor tracking of temperature, UV exposure, and expiration date of a drug throughout four time intervals, and a process for adjusting the dosage of the drug;

FIG. 13 is a schematic view of one embodiment of a drug holder having a drug status indicator disposed thereon;

FIG. 14 is a schematic view of one embodiment of a drug administration device having the drug holder of FIG. 13 disposed therein;

FIG. 15A is a schematic view of one embodiment of a drug holder having a label disposed thereon, showing the label in a first state;

FIG. 15B is a schematic view of the drug holder in FIG. 15A, showing the label in a second state;

FIG. 16 is a schematic view of one embodiment of a housing for a drug administration device having a label disposed thereon; and

FIG. 17 is a schematic view of one embodiment of a reader configured to read the label of FIG. 16.

### DETAILED DESCRIPTION

Certain exemplary embodiments will now be described to provide an overall understanding of the principles of the structure, function, manufacture, and use of the devices, systems, and methods disclosed herein. One or more examples of these embodiments are illustrated in the accompanying drawings. A person skilled in the art will understand that the devices, systems, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. A person skilled in the art will appreciate that a dimension may not be a precise value but nevertheless be considered to be at about that value due to any number of factors such as manufacturing tolerances and sensitivity of measurement equipment. Sizes and shapes of the systems and devices, and the components thereof, can depend at least on the size and shape of components with which the systems and devices will be used.

Examples of various types of drug administration devices, namely: an autoinjector 100, an infusion pump 200, an inhaler 300, and a nasal spray device 400, are described below with reference to the hereinbefore referenced figures.

### Autoinjector

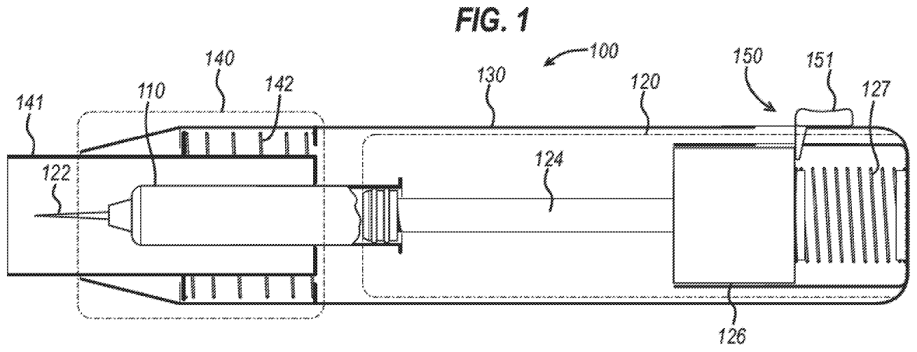

FIG. 1 is a schematic exemplary view of a first type of drug delivery device, namely an injection device, in this example an autoinjector 100, useable with embodiments described herein. The autoinjector 100 comprises a drug holder 110 which retains a drug to be dispensed and a dispensing mechanism 120 which is configured to dispense a drug from the drug holder 110 so that it can be administered to a patient. The drug holder 110 is typically in the form of a container which contains the drug, for example it may be provided in the form of a syringe or a vial, or be any other suitable container which can hold the drug. The autoinjector 100 comprises a discharge nozzle 122, for example a needle of a syringe, which is provided at a distal end of the drug holder 110. The dispensing mechanism 120 comprises a drive element 124, which itself may also comprise a piston and/or a piston rod, and drive mechanism 126. The dispensing mechanism 120 is located proximal to the end of the drug holder 110 and towards the proximal end of the autoinjector 100.

The autoinjector 100 comprises a housing 130 which contains the drug holder 110, drive element 124 and drive mechanism 126 within the body of the housing 130, as well as containing the discharge nozzle 122, which, prior to injection, would typically be contained fully within the housing, but which would extend out of the housing 130 during an injection sequence to deliver the drug. The dispensing mechanism 120 is arranged so that the drive element 124 is advanced through the drug holder 110 in order to dispense the drug through the discharge nozzle 122, thereby allowing the autoinjector to administer a drug retained in drug holder 110 to a patient. In some instances, a user may advance the drive element 124 through the drug holder 110 manually. In other instances, the drive mechanism 126 may include a stored energy source 127 which advances the drive element 124 without user assistance. The stored energy source 127 may include a resilient biasing member such as a spring, or a pressurized gas, or electronically powered motor and/or gearbox.

The autoinjector 100 includes a dispensing mechanism protection mechanism 140. The dispensing mechanism protection mechanism 140 typically has two functions. Firstly, the dispensing mechanism protection mechanism 140 can function to prevent access to the discharge nozzle 122 prior to and after injection. Secondly, the autoinjector 100 can function, such that when put into an activated state, e.g., the dispensing mechanism protection mechanism 140 is moved to an unlocked position, the dispensing mechanism 120 can be activated.

The protection mechanism 140 covers at least a part of the discharge nozzle 122 when the drug holder 110 is in its retracted position proximally within the housing 130. This is to impede contact between the discharge nozzle 122 and a user. Alternatively, or in addition, the protection mechanism 140 is itself configured to retract proximally to expose the discharge nozzle 122 so that it can be brought into contact with a patient. The protection mechanism 140 comprises a shield member 141 and return spring 142. Return spring 142 acts to extend the shield member 141 from the housing 130, thereby covering the discharge nozzle 122 when no force is applied to the distal end of the protection mechanism 140. If a user applies a force to the shield member 141 against the action of the return spring 142 to overcome the bias of the return spring 142, the shield member 141 retracts within the housing 130, thereby exposing the discharge nozzle 122. The protection mechanism 140 may alternatively, or in addition, comprise an extension mechanism (not shown) for extending the discharge nozzle 122 beyond the housing 130, and may further comprise a retracting mechanism (not shown) for retracting the discharge nozzle 122 within the housing 130. The protection mechanism 140 may alternatively, or in addition, comprise a housing cap and/or discharge nozzle boot, which can be attached to the autoinjector 100. Removal of the housing cap would typically also remove the discharge nozzle boot from the discharge nozzle 122.

The autoinjector 100 also includes a trigger 150. The trigger 150 comprises a trigger button 151 which is located on an external surface of the housing 130 so that it is accessible by a user of the autoinjector 100. When the trigger 150 is pressed by a user, it acts to release the drive mechanism 126 so that, via the drive element 124, the drug is then driven out of the drug holder 110 via the discharge nozzle 122.

The trigger 150 may also cooperate with the shield member 141 in such a way that the trigger 150 is prevented from being activated until the shield member 141 has been retracted proximally sufficiently into the housing 130 into an unlocked position, for example by pushing a distal end of the shield member 141 against the skin of a patient. When this has been done, the trigger 150 becomes unlocked, and the autoinjector 100 is activated such that the trigger 150 can be depressed and the injection and/or drug delivery sequence is then initiated. Alternatively, retraction of the shield member 141 alone in a proximal direction into the housing 130 can act to activate the drive mechanism 126 and initiate the injection and/or drug delivery sequence. In this way, the autoinjector 100 has device operation prevention mechanism which prevents dispensing of the drug by, for example, preventing accidental release of the dispensing mechanism 120 and/or accidental actuation of the trigger 150.

While the foregoing description relates to one example of an autoinjector, this example is presented purely for illustration, the present invention is not limited solely to such an autoinjector. A person skilled in the art understands that various modifications to the described autoinjector may be implemented within the scope of the present disclosure.

Autoinjectors of the present disclosure can be used to administer any of a variety of drugs, such as any of epinephrine, Rebif, Enbrel, Aranesp, atropine, pralidoxime chloride, and diazepam.

### Infusion Pump

In other circumstances, patients can require precise, continuous delivery of medication or medication delivery on a regular or frequent basis at set periodic intervals. Infusion pumps can provide such controlled drug infusion, by facilitating the administering of the drug at a precise rate that keeps the drug concentration within a therapeutic margin, without requiring frequent attention by a healthcare professional or the patient.

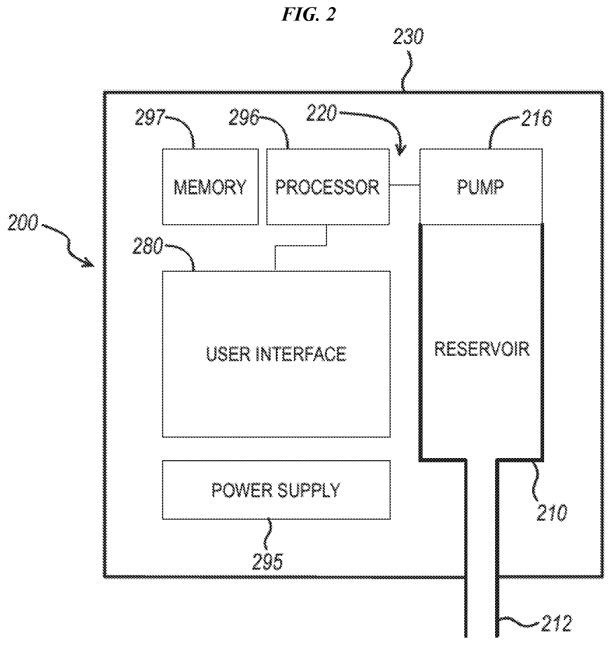

FIG. 2 is a schematic exemplary view of a second type of drug delivery device, namely an infusion pump 200, useable with the embodiments described herein. The infusion pump 200 comprises a drug holder 210 in the form of a reservoir for containing a drug to be delivered, and a dispensing mechanism 220 comprising a pump 216 adapted to dispense a drug contained in the reservoir, so that the drug can be delivered to a patient. These components of the infusion pump are located within housing 230. The dispensing mechanism 220 further comprises an infusion line 212. The drug is delivered from the reservoir upon actuation of the pump 216 via the infusion line 212, which may take the form of a cannula. The pump 216 may take the form of an elastomeric pump, a peristaltic pump, an osmotic pump, or a motor-controlled piston in a syringe. Typically, the drug is delivered intravenously, although subcutaneous, arterial and epidural infusions may also be used.

Infusion pumps of the present disclosure can be used to administer any of a variety of drugs, such as any of insulin, antropine sulfate, avibactam sodium, bendamustine hydrochloride, carboplatin, daptomycin, epinephrine, levetiracetam, oxaliplatin, paclitaxel, pantoprazole sodium, treprostinil, vasopressin, voriconazole, and zoledronic acid.

The infusion pump 200 further comprises control circuitry, for example a processor 296 in addition to a memory 297 and a user interface 280, which together provide a triggering mechanism and/or dosage selector for the pump 200. The user interface 280 may be implemented by a display screen located on the housing 230 of the infusion pump 200. The control circuitry and user interface 280 can be located within the housing 230, or external thereto and communicate via a wired or wireless interface with the pump 216 to control its operation.

Actuation of the pump 216 is controlled by the processor 296 which is in communication with the pump 216 for controlling the pump's operation. The processor 296 may be programmed by a user (e.g., patient or healthcare professional), via a user interface 280. This enables the infusion pump 200 to deliver the drug to a patient in a controlled manner. The user can enter parameters, such as infusion duration and delivery rate. The delivery rate may be set by the user to a constant infusion rate or as set intervals for periodic delivery, typically within pre-programmed limits. The programmed parameters for controlling the pump 216 are stored in and retrieved from the memory 297 which is in communication with the processor 296. The user interface 280 may take the form of a touch screen or a keypad.

A power supply 295 provides power to the pump 216, and may take the form of an energy source which is integral to the pump 216 and/or a mechanism for connecting the pump 216 to an external source of power.

The infusion pump 200 may take on a variety of different physical forms depending on its designated use. It may be a stationary, non-portable device, e.g., for use at a patient's bedside, or it may be an ambulatory infusion pump which is designed to be portable or wearable. An integral power supply 295 is particularly beneficial for ambulatory infusion pumps.

While the foregoing description relates to one example of an infusion pump, this example is provided purely for illustration. The present disclosure is not limited to such an infusion pump. A person skilled in the art understands that various modifications to the described infusion pump may be implemented within the scope of the present disclosure. For example, the processor may be pre-programmed, such that it is not necessary for the infusion pump to include a user interface.

### Inhaler

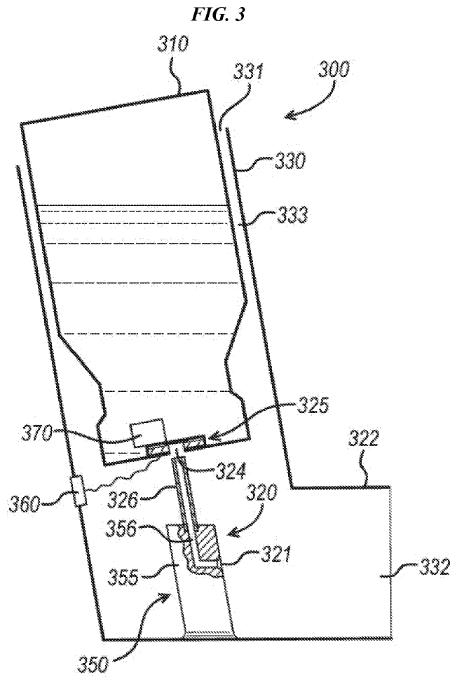

FIG. 3 is a schematic view of a third type of drug administration device, namely an inhaler 300. Inhaler 300 includes a drug holder 310 in the form of a canister. The drug holder 310 contains a drug that would typically be in solution or suspension with a suitable carrier liquid. The inhaler 300 further comprises a dispensing mechanism 320, which includes a pressurized gas for pressurizing the drug holder 310, a valve 325 and nozzle 321. The valve 325 forms an outlet of the drug holder 310. The valve 325 comprises a narrow opening 324 formed in the drug holder 310 and a movable element 326 that controls the opening 324. When the movable element 326 is in a resting position, the valve 325 is in a closed or unactuated state in which the opening 324 is closed and the drug holder 310 is sealed. When the movable element 326 is actuated from the resting position to an actuated position, the valve 325 is actuated into an open state in which the opening 324 is open. Actuation of the movable element 326 from the resting position to the actuated position comprises moving the movable element 326 into the drug holder 310. The movable element 326 is resiliently biased into the resting position. In the open state of the valve 325, the pressurized gas propels the drug in solution or suspension with the suitable liquid out of the drug holder 310 through the opening 324 at high speed. The high speed passage of the liquid through the narrow opening 324 causes the liquid to be atomized, that is, to transform from a bulk liquid into a mist of fine droplets of liquid and/or into a gas cloud. A patient may inhale the mist of fine droplets and/or the gas cloud into a respiratory passage. Hence, the inhaler 300 is capable of delivering a drug retained within the drug holder 310 into a respiratory passage of a patient.

The drug holder 310 is removably held within a housing 330 of the inhaler 300. A passage 333 formed in the housing 330 connects a first opening 331 in the housing 330 and a second opening 332 in the housing 330. The drug holder 310 is received within the passage 333. The drug holder 310 is slidably insertable through the first opening 331 of the housing 330 into the passage 333. The second opening 332 of the housing 330 forms a mouthpiece 322 configured to be placed in a patient's mouth or a nosepiece configured to be placed in a patient's nostril, or a mask configured to be placed over the patient's mouth and nose. The drug holder 310, the first opening 331 and the passage 333 are sized such that air can flow through the passage 333, around the drug holder 310, between the first opening 331 and the second opening 332. The inhaler 300 may be provided with a dispensing mechanism protection mechanism 140 in the form of a cap (not shown) which can be fitted to the mouthpiece 322.

Inhaler 300 further comprises a trigger 350 including a valve actuation feature 355 configured to actuate the valve 325 when the trigger 350 is activated. The valve actuation feature 355 is a projection of the housing 330 into the passage 333. The drug holder 310 is slidably movable within the passage 333 from a first position into a second position. In the first position, an end of the movable element 326 in the resting position abuts the valve actuation feature 355. In the second position, the drug holder 310 can be displaced towards the valve actuation feature 355 such that the valve actuation feature 355 moves the movable element 326 into the drug holder 310 to actuate the valve 325 into the open state. The user's hand provides the necessary force to move the drug holder 310 from the first position to the second position against the resiliently biased movable element 326. The valve actuation feature 355 includes an inlet 356, which is connected to the nozzle 321. The inlet 356 of the valve actuation feature 355 is sized and positioned to couple to the opening 324 of the valve 325 such that the ejected mist of droplets and/or gas cloud can enter the inlet 356 and exit from the nozzle 321 into the passage 333. The nozzle 321 assists in the atomization of the bulk liquid into the mist of droplets and/or gas cloud.

The valve 325 provides a metering mechanism 370. The metering mechanism 370 is configured to close the valve after a measured amount of liquid, and therefore, drug, has passed through the opening 324. This allows a controlled dose to be administered to the patient. Typically, the measured amount of liquid is pre-set, however, the inhaler 300 may be equipped with a dosage selector 360 that is user operable to change the defined amount of liquid.

While the foregoing description relates to one particular example of an inhaler, this example is purely illustrative. The description should not be seen as limited only to such an inhaler. A person skilled in the art understands that numerous other types of inhaler and nebulizers may be used with the present disclosure. For example, the drug may be in a powdered form, the drug may be in liquid form, or the drug may be atomized by other forms of dispensing mechanism 320 including ultrasonic vibration, compressed gas, a vibrating mesh, or a heat source.

The inhalers of the present disclosure can be used to administer any of a variety of drugs, such as any of mometasone, fluticasone, ciclesonide, budesonide, beclomethasone, vilanterol, salmeterol, formoterol, umeclidinium, glycopyrrolate, tiotropium, aclidinium, indacaterol, salmeterol, and olodaterol.

### Nasal Spray Device

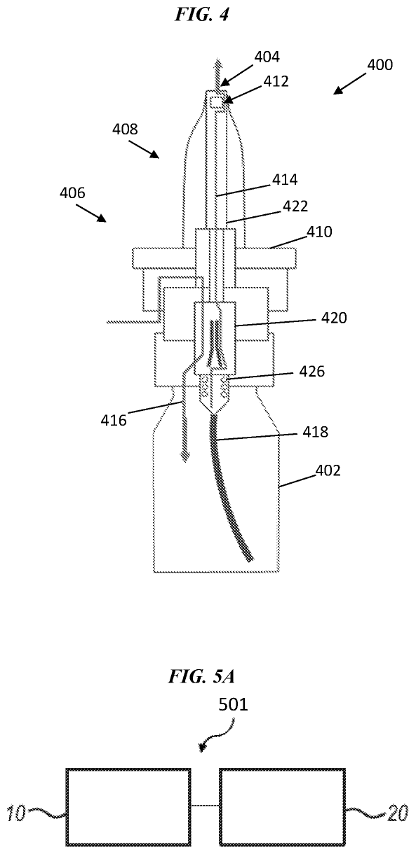

FIG. 4 is a schematic view of a fourth type of drug administration device, namely a nasal spray device 400. The nasal spray device 400 is configured to expel a drug into a nose of a patient. The nasal spray device 400 includes a drug holder 402 configured to contain a drug therein for delivery from the device 400 to a patient. The drug holder 102 can have a variety of configurations, such as a bottle reservoir, a cartridge, a vial (as in this illustrated embodiment), a blow-fill-seal (BFS) capsule, a blister pack, etc. In an exemplary embodiment, the drug holder 402 is a vial. An exemplary vial is formed of one or more materials, e.g., glass, polymer(s), etc. In some embodiments, a vial can be formed of glass. In other embodiments, a vial can be formed of one or more polymers. In yet other embodiments, different portions of a vial can be formed of different materials. An exemplary vial can include a variety of features to facilitate sealing and storing a drug therein, as described herein and illustrated in the drawings. However, a person skilled in the art will appreciate that the vials can include only some of these features and/or can include a variety of other features known in the art. The vials described herein are merely intended to represent certain exemplary embodiments.

An opening 404 of the nasal spray device 400 through which the drug exits the nasal spray device 400 is formed in in a dispensing head 406 of the nasal spray device 400 in a tip 408 of the dispensing head 406. The tip 408 is configured to be inserted into a nostril of a patient. In an exemplary embodiment, the tip 408 is configured to be inserted into a first nostril of the patient during a first stage of operation of the nasal spray device 400 and into a second nostril of the patient during a second stage of operation of the nasal spray device 400. The first and second stages of operation involve two separate actuations of the nasal spray device 400, a first actuation corresponding to a first dose of the drug being delivered and a second actuation corresponding to a second dose of the drug being delivered. In some embodiments, the nasal spray device 400 is configured to be actuated only once to deliver one nasal spray. In some embodiments, the nasal spray device 400 is configured to be actuated three or more times to deliver three or more nasal sprays, e.g., four, five, six, seven, eight, nine, ten, etc.

The dispensing head 406 includes a depth guide 410 configured to contact skin of the patient between the patient's first and second nostrils, such that a longitudinal axis of the dispensing head 406 is substantially aligned with a longitudinal axis of the nostril in which the tip 408 is inserted. A person skilled in the art will appreciate that the longitudinal axes may not be precisely aligned but nevertheless be considered to be substantially aligned due to any number of factors, such as manufacturing tolerances and sensitivity of measurement equipment.

In an exemplary embodiment, as in FIG. 4, the dispensing head 406 has a tapered shape in which the dispensing head 406 has a smaller diameter at its distal end than at its proximal end where the opening 404 is located. The opening 404 having a relatively small diameter facilitates spray of the drug out of the opening 404, as will be appreciated by a person skilled in the art. A spray chamber 412 through which the drug is configured to pass before exiting the opening 404 is located within a proximal portion of the tapered dispensing head 406, distal to the opening 404. When the drug passes through the spray chamber 412 at speed, the spray chamber 412 facilitates production of a fine mist that exits through the opening 404 with a consistent spray pattern. Arrow 414 in FIG. 4 illustrates a path of travel of the drug from the drug holder 402 and out of the opening 404.

In some embodiments, the dispensing head 406 can include two tips 408 each having an opening 404 therein such that the nasal spray device 400 is configured to simultaneously deliver doses of drug into two nostrils in response to a single actuation.

The dispensing head 406 is configured to be pushed toward the drug holder 402, e.g., depressed by a user pushing down on the depth guide 410, to actuate the nasal spray device 400. In other words, the dispensing head 406 is configured as an actuator to be actuated to drive the drug from the drug holder 402 and out of the nasal spray device 400. In an exemplary embodiment, the nasal spray device 400 is configured to be self-administered such that the user who actuates the nasal spray device 400 is the patient receiving the drug from the nasal spray device 400, although another person can actuate the nasal spray device 400 for delivery into another person.

The actuation, e.g., depressing, of the dispensing head 406 is configured to cause venting air to enter the drug holder 402, as shown by arrow 416 in FIG. 4. The air entering the drug holder 402 displaces drug in the drug holder through a tube 418 and then into a metering chamber 420, which displaces drug proximally through a cannula 422, through the spray chamber 412, and then out of the opening 404. In response to release of the dispensing head 406, e.g., a user stops pushing downward on the dispensing head 406, a bias spring 426 causes the dispensing head 406 to return to its default, resting position to position the dispensing head 406 relative to the drug holder 402 for a subsequent actuation and drug delivery.

While the foregoing description relates to one particular example of a nasal spray device, this example is purely illustrative. The description should not be seen as limited only to such a nasal spray device. A person skilled in the art understands that the nasal spray device 400 can include different features in different embodiments depending upon various requirements. For example, the nasal spray device 400 can lack the depth guide 410 and/or may include any one or more of a device indicator, a sensor, a communications interface, a processor, a memory, and a power supply.

The nasal spray devices of the present disclosure can be used to administer any of a variety of drugs, such as any of ketamine (e.g., Ketalar®), esketamine (e.g., Spravato®, Ketanest®, and Ketanest-S®), naloxone (e.g., Narcan®), and sumatriptan (e.g., Imitrex®).

### Drug Administration Device

As will be appreciated from the foregoing, various components of drug delivery devices are common to all such devices. These components form the essential components of a universal drug administration device. A drug administration device delivers a drug to a patient, where the drug is provided in a defined dosage form within the drug administration device.

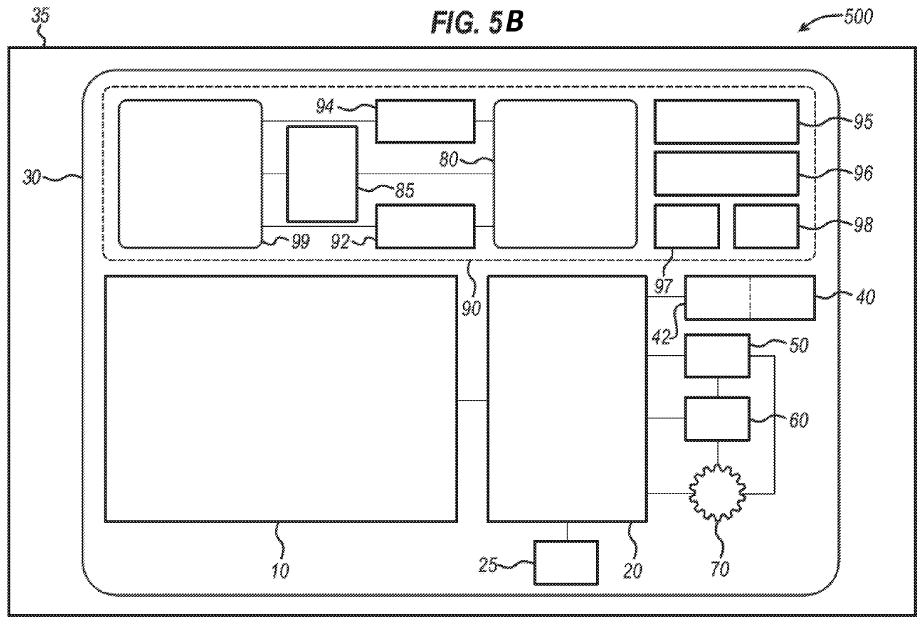

FIG. 5A is a generalized schematic view of such a universal drug administration device 501, and FIG. 5B is an exemplary embodiment of such a universal drug administration device 500. Examples of the universal drug administration device 500 include injection devices (e.g., autoinjectors, jet injectors, and infusion pumps), nasal spray devices, and inhalers.

As shown in FIG. 5A, drug administration device 501 includes in general form the features of a drug holder 10 and a dispensing mechanism 20. The drug holder 10 holds a drug in a dosage form to be administered. The dispensing mechanism 20 is configured to release the dosage form from the drug holder 10 so that the drug can be administered to a patient.

FIG. 5B shows a further universal drug administration device 500 which includes a number of additional features. A person skilled in the art understands that these additional features are optional for different embodiments, and can be utilized in a variety of different combinations such that the additional features may be present or may be omitted from a given embodiment of a particular drug administration device, depending upon requirements, such as the type of drug, dosage form of the drug, medical indication being treated with the drug, safety requirements, whether the device is powered, whether the device is portable, whether the device is used for self-administration, and many other requirements which will be appreciated by a person skilled in the art. Similar to the universal device of FIG. 5A, the drug administration device 500 comprises a housing 30 which accommodates the drug holder 10 and dispensing mechanism 20.

The device 500 is provided with a triggering mechanism 50 for initiating the release of the drug from the drug holder 10 by the dispensing mechanism 20. The device 500 includes the feature of a metering/dosing mechanism 70 which measures out a set dose to be released from the drug holder 10 via the dispensing mechanism 20. In this manner, the drug administration device 500 can provide a known dose of determined size. The device 500 comprises a dosage selector 60 which enables a user to set the dose volume of drug to be measured out by the metering mechanism 70. The dose volume can be set to one specific value of a plurality of predefined discrete dose volumes, or any value of predefined dose volume within a range of dose volumes.

The device 500 can comprise a device operation prevention mechanism 40 or 25 which when in a locked state will prevent and/or stop the dispensing mechanism 20 from releasing the drug out of the drug holder 10, and when in an unlocked state will permit the dispensing mechanism 20 to release the drug dosage from out of the drug holder 10. This can prevent accidental administration of the drug, for example to prevent dosing at an incorrect time, or for preventing inadvertent actuation. The device 500 also includes a dispensing mechanism protection mechanism 42 which prevents access to at least a part of the dispensing mechanism 20, for example for safety reasons. Device operation prevention mechanism 40 and dispensing mechanism protection mechanism 42 may be the same component.

The device 500 can include a device indicator 85 which is configured to present information about the status of the drug administration device and/or the drug contained therein. The device indicator 85 may be a visual indicator, such as a display screen, or an audio indicator. The device 500 includes a user interface 80 which can be configured to present a user of the device 500 with information about the device 500 and/or to enable the user to control the device 500. The device 500 includes a device sensor 92 which is configured to sense information relating to the drug administration device and/or the drug contained therein, for example dosage form and device parameters. As an example, in embodiments which include a metering mechanism 70 and a dosage selector 60, the embodiment may further include one or more device sensors 92 configured to sense one or more of: the dose selected by a user using dosage selector 60, the dose metered by the metering mechanism 70 and the dose dispensed by the dispensing mechanism 20. Similarly, an environment sensor 94 is provided which is configured to sense information relating to the environment in which the device 500 is present, such as the temperature of the environment, the temperature of the environment, location, and time. There may be a dedicated location sensor 98 which is configured to determine the geographical location of the device 500, e.g., via satellite position determination, such as GPS. The device 500 also includes a communications interface 99 which can communicate externally data which has been acquired from the various sensors about the device and/or drug.

If required, the device 500 comprises a power supply 95 for delivering electrical power to one or more electrical components of the device 500. The power supply 95 can be a source of power which is integral to device 500 and/or a mechanism for connecting device 500 to an external source of power. The drug administration device 500 also includes a device computer system 90 including processor 96 and memory 97 powered by the power supply 95 and in communication with each other, and optionally with other electrical and control components of the device 500, such as the environment sensor 94, location sensor 98, device sensor 92, communications interface 99, and/or indicator 85. The processor 96 is configured to obtain data acquired from the environment sensor 94, device sensor 92, communications interface 99, location sensor 98, and/or user interface 80 and process it to provide data output, for example to indicator 85 and/or to communications interface 99.

In some embodiments, the drug administration device 500 is enclosed in packaging 35. The packaging 35 may further include a combination of a processor 96, memory 97, user interface 80, device indicator 85, device sensor 92, location sensor 98 and/or environment sensors 94 as described herein, and these may be located externally on the housing of the device 500.

A person skilled in the art will appreciate that the universal drug administration device 500 comprising the drug holder 10 and dispensing mechanism 20 can be provided with a variety of the optional features described above, in a number of different combinations. Moreover, the drug administration device 500 can include more than one drug holder 10, optionally with more than one dispensing mechanism 20, such that each drug holder has its own associated dispensing mechanism 20.

### Drug Dosage Forms

Conventionally, drug administration devices utilize a liquid dosage form. It will be appreciated, however that other dosage forms are available.

One such common dosage form is a tablet. The tablet may be formed from a combination of the drug and an excipient that are compressed together. Other dosage forms are pastes, creams, powders, ear drops, and eye drops.

Further examples of drug dosage forms include dermal patches, drug eluting stents and intrauterine devices. In these examples, the body of the device comprises the drug and may be configured to allow the release of the drug under certain circumstances. For example, a dermal patch may comprise a polymeric composition containing the drug. The polymeric composition allows the drug to diffuse out of the polymeric composition and into the skin of the patient. Drug eluting stents and intrauterine devices can operate in an analogous manner. In this way, the patches, stents and intrauterine devices may themselves be considered drug holders with an associated dispensing mechanism.

Any of these dosage forms can be configured to have the drug release initiated by certain conditions. This can allow the drug to be released at a desired time or location after the dosage form has been introduced into the patient. In particular, the drug release may be initiated by an external stimulus. Moreover, these dosage forms can be contained prior to administration in a housing, which may be in the form of packaging. This housing may contain some of the optional features described above which are utilized with the universal drug administration device 500.

The drug administered by the drug administration devices of the present disclosure can be any substance that causes a change in an organism's physiology or psychology when consumed. Examples of drugs that the drug administration devices of the present disclosure can administer include 5-alpha-reductase inhibitors, 5-aminosalicylates, 5HT3 receptor antagonists, ACE inhibitors with calcium channel blocking agents, ACE inhibitors with thiazides, adamantane antivirals, adrenal cortical steroids, adrenal corticosteroid inhibitors, adrenergic bronchodilators, agents for hypertensive emergencies, agents for pulmonary hypertension, aldosterone receptor antagonists, alkylating agents, allergenics, alpha-glucosidase inhibitors, alternative medicines, amebicides, aminoglycosides, aminopenicillins, aminosalicylates, AMPA receptor antagonists, amylin analogs, analgesic combinations, analgesics, androgens and anabolic steroids, Angiotensin Converting Enzyme Inhibitors, angiotensin II inhibitors with calcium channel blockers, angiotensin II inhibitors with thiazides, angiotensin receptor blockers, angiotensin receptor blockers and neprilysin inhibitors, anorectal preparations, anorexiants, antacids, anthelmintics, anti-angiogenic ophthalmic agents, anti-CTLA-4 monoclonal antibodies, anti-infectives, anti-PD-1 monoclonal antibodies, antiadrenergic agents (central) with thiazides, antiadrenergic agents (peripheral) with thiazides, antiadrenergic agents, centrally acting, antiadrenergic agents, peripherally acting, antiandrogens, antianginal agents, antiarrhythmic agents, antiasthmatic combinations, antibiotics/antineoplastics, anticholinergic antiemetics, anticholinergic antiparkinson agents, anticholinergic bronchodilators, anticholinergic chronotropic agents, anticholinergics/antispasmodics, anticoagulant reversal agents, anticoagulants, anticonvulsants, antidepressants, antidiabetic agents, antidiabetic combinations, antidiarrheals, antidiuretic hormones, antidotes, antiemetic/antivertigo agents, antifungals, antigonadotropic agents, antigout agents, antihistamines, antihyperlipidemic agents, antihyperlipidemic combinations, antihypertensive combinations, antihyperuricemic agents, antimalarial agents, antimalarial combinations, antimalarial quinolones, antimanic agents, antimetabolites, antimigraine agents, antineoplastic combinations, antineoplastic detoxifying agents, antineoplastic interferons, antineoplastics, antiparkinson agents, antiplatelet agents, antipseudomonal penicillins, antipsoriatics, antipsychotics, antirheumatics, antiseptic and germicides, antithyroid agents, antitoxins and antivenins, antituberculosis agents, antituberculosis combinations, antitussives, antiviral agents, antiviral boosters, antiviral combinations, antiviral interferons, anxiolytics, sedatives, and hypnotics, aromatase inhibitors, atypical antipsychotics, azole antifungals, bacterial vaccines, barbiturate anticonvulsants, barbiturates, BCR-ABL tyrosine kinase inhibitors, benzodiazepine anticonvulsants, benzodiazepines, beta blockers with calcium channel blockers, beta blockers with thiazides, beta-adrenergic blocking agents, beta-lactamase inhibitors, bile acid sequestrants, biologicals, bisphosphonates, bone morphogenetic proteins, bone resorption inhibitors, bronchodilator combinations, bronchodilators, calcimimetics, calcineurin inhibitors, calcitonin, calcium channel blocking agents, carbamate anticonvulsants, carbapenems, carbapenems/beta-lactamase inhibitors, carbonic anhydrase inhibitor anticonvulsants, carbonic anhydrase inhibitors, cardiac stressing agents, cardioselective beta blockers, cardiovascular agents, catecholamines, cation exchange resins, CD20 monoclonal antibodies, CD30 monoclonal antibodies, CD33 monoclonal antibodies, CD38 monoclonal antibodies, CD52 monoclonal antibodies, CDK 4/6 inhibitors, central nervous system agents, cephalosporins, cephalosporins/beta-lactamase inhibitors, cerumenolytics, CFTR combinations, CFTR potentiators, CGRP inhibitors, chelating agents, chemokine receptor antagonist, chloride channel activators, cholesterol absorption inhibitors, cholinergic agonists, cholinergic muscle stimulants, cholinesterase inhibitors, CNS stimulants, coagulation modifiers, colony stimulating factors, contraceptives, corticotropin, coumarins and indandiones, cox-2 inhibitors, decongestants, dermatological agents, diagnostic radiopharmaceuticals, diarylquinolines, dibenzazepine anticonvulsants, digestive enzymes, dipeptidyl peptidase 4 inhibitors, diuretics, dopaminergic antiparkinsonism agents, drugs used in alcohol dependence, echinocandins, EGFR inhibitors, estrogen receptor antagonists, estrogens, expectorants, factor Xa inhibitors, fatty acid derivative anticonvulsants, fibric acid derivatives, first generation cephalosporins, fourth generation cephalosporins, functional bowel disorder agents, gallstone solubilizing agents, gamma-aminobutyric acid analogs, gamma-aminobutyric acid reuptake inhibitors, gastrointestinal agents, general anesthetics, genitourinary tract agents, GI stimulants, glucocorticoids, glucose elevating agents, glycopeptide antibiotics, glycoprotein platelet inhibitors, glycylcyclines, gonadotropin releasing hormones, gonadotropin-releasing hormone antagonists, gonadotropins, group I antiarrhythmics, group II antiarrhythmics, group III antiarrhythmics, group IV antiarrhythmics, group V antiarrhythmics, growth hormone receptor blockers, growth hormones, guanylate cyclase-C agonists, H. pylori eradication agents, H2 antagonists, hedgehog pathway inhibitors, hematopoietic stem cell mobilizer, heparin antagonists, heparins, HER2 inhibitors, herbal products, histone deacetylase inhibitors, hormones, hormones/antineoplastics, hydantoin anticonvulsants, hydrazide derivatives, illicit (street) drugs, immune globulins, immunologic agents, immunostimulants, immunosuppressive agents, impotence agents, in vivo diagnostic biologicals, incretin mimetics, inhaled anti-infectives, inhaled corticosteroids, inotropic agents, insulin, insulin-like growth factors, integrase strand transfer inhibitor, interferons, interleukin inhibitors, interleukins, intravenous nutritional products, iodinated contrast media, ionic iodinated contrast media, iron products, ketolides, laxatives, leprostatics, leukotriene modifiers, lincomycin derivatives, local injectable anesthetics, local injectable anesthetics with corticosteroids, loop diuretics, lung surfactants, lymphatic staining agents, lysosomal enzymes, macrolide derivatives, macrolides, magnetic resonance imaging contrast media, mast cell stabilizers, medical gas, meglitinides, metabolic agents, methylxanthines, mineralocorticoids, minerals and electrolytes, miscellaneous agents, miscellaneous analgesics, miscellaneous antibiotics, miscellaneous anticonvulsants, miscellaneous antidepressants, miscellaneous antidiabetic agents, miscellaneous antiemetics, miscellaneous antifungals, miscellaneous antihyperlipidemic agents, miscellaneous antihypertensive combinations, miscellaneous antimalarials, miscellaneous antineoplastics, miscellaneous antiparkinson agents, miscellaneous antipsychotic agents, miscellaneous antituberculosis agents, miscellaneous antivirals, miscellaneous anxiolytics, sedatives and hypnotics, miscellaneous bone resorption inhibitors, miscellaneous cardiovascular agents, miscellaneous central nervous system agents, miscellaneous coagulation modifiers, miscellaneous diagnostic dyes, miscellaneous diuretics, miscellaneous genitourinary tract agents, miscellaneous GI agents, miscellaneous hormones, miscellaneous metabolic agents, miscellaneous ophthalmic agents, miscellaneous otic agents, miscellaneous respiratory agents, miscellaneous sex hormones, miscellaneous topical agents, miscellaneous uncategorized agents, miscellaneous vaginal agents, mitotic inhibitors, monoamine oxidase inhibitors, mouth and throat products, mTOR inhibitors, mucolytics, multikinase inhibitors, muscle relaxants, mydriatics, narcotic analgesic combinations, narcotic analgesics, nasal anti-infectives, nasal antihistamines and decongestants, nasal lubricants and irrigations, nasal preparations, nasal steroids, natural penicillins, neprilysin inhibitors, neuraminidase inhibitors, neuromuscular blocking agents, neuronal potassium channel openers, next generation cephalosporins, nicotinic acid derivatives, NK1 receptor antagonists, NNRTIs, non-cardioselective beta blockers, non-iodinated contrast media, non-ionic iodinated contrast media, non-sulfonylureas, Nonsteroidal anti-inflammatory drugs, NS5A inhibitors, nucleoside reverse transcriptase inhibitors (NRTIs), nutraceutical products, nutritional products, ophthalmic anesthetics, ophthalmic anti-infectives, ophthalmic anti-inflammatory agents, ophthalmic antihistamines and decongestants, ophthalmic diagnostic agents, ophthalmic glaucoma agents, ophthalmic lubricants and irrigations, ophthalmic preparations, ophthalmic steroids, ophthalmic steroids with anti-infectives, ophthalmic surgical agents, oral nutritional supplements, other immunostimulants, other immunosuppressants, otic anesthetics, otic anti-infectives, otic preparations, otic steroids, otic steroids with anti-infectives, oxazolidinedione anticonvulsants, oxazolidinone antibiotics, parathyroid hormone and analogs, PARP inhibitors, PCSK9 inhibitors, penicillinase resistant penicillins, penicillins, peripheral opioid receptor antagonists, peripheral opioid receptor mixed agonists/antagonists, peripheral vasodilators, peripherally acting antiobesity agents, phenothiazine antiemetics, phenothiazine antipsychotics, phenylpiperazine antidepressants, phosphate binders, PI3K inhibitors, plasma expanders, platelet aggregation inhibitors, platelet-stimulating agents, polyenes, potassium sparing diuretics with thiazides, potassium-sparing diuretics, probiotics, progesterone receptor modulators, progestins, prolactin inhibitors, prostaglandin D2 antagonists, protease inhibitors, protease-activated receptor-1 antagonists, proteasome inhibitors, proton pump inhibitors, psoralens, psychotherapeutic agents, psychotherapeutic combinations, purine nucleosides, pyrrolidine anticonvulsants, quinolones, radiocontrast agents, radiologic adjuncts, radiologic agents, radiologic conjugating agents, radiopharmaceuticals, recombinant human erythropoietins, renin inhibitors, respiratory agents, respiratory inhalant products, rifamycin derivatives, salicylates, sclerosing agents, second generation cephalosporins, selective estrogen receptor modulators, selective immunosuppressants, selective phosphodiesterase-4 inhibitors, selective serotonin reuptake inhibitors, serotonin-norepinephrine reuptake inhibitors, serotoninergic neuroenteric modulators, sex hormone combinations, sex hormones, SGLT-2 inhibitors, skeletal muscle relaxant combinations, skeletal muscle relaxants, smoking cessation agents, somatostatin and somatostatin analogs, spermicides, statins, sterile irrigating solutions, streptogramins, streptomyces derivatives, succinimide anticonvulsants, sulfonamides, sulfonylureas, synthetic ovulation stimulants, tetracyclic antidepressants, tetracyclines, therapeutic radiopharmaceuticals, therapeutic vaccines, thiazide diuretics, thiazolidinediones, thioxanthenes, third generation cephalosporins, thrombin inhibitors, thrombolytics, thyroid drugs, TNF alfa inhibitors, tocolytic agents, topical acne agents, topical agents, topical allergy diagnostic agents, topical anesthetics, topical anti-infectives, topical anti-rosacea agents, topical antibiotics, topical antifungals, topical antihistamines, topical antineoplastics, topical antipsoriatics, topical antivirals, topical astringents, topical debriding agents, topical depigmenting agents, topical emollients, topical keratolytics, topical non-steroidal anti-inflammatories, topical photochemotherapeutics, topical rubefacient, topical steroids, topical steroids with anti-infectives, transthyretin stabilizers, triazine anticonvulsants, tricyclic antidepressants, trifunctional monoclonal antibodies, ultrasound contrast media, upper respiratory combinations, urea anticonvulsants, urea cycle disorder agents, urinary anti-infectives, urinary antispasmodics, urinary pH modifiers, uterotonic agents, vaccine combinations, vaginal anti-infectives, vaginal preparations, vasodilators, vasopressin antagonists, vasopressors, VEGF/VEGFR inhibitors, viral vaccines, viscosupplementation agents, vitamin and mineral combinations, vitamins, or VMAT2 inhibitors. The drug administration devices of the present disclosure may administer a drug selected from epinephrine, Rebif, Enbrel, Aranesp, atropine, pralidoxime chloride, diazepam, insulin, antropine sulfate, avibactam sodium, bendamustine hydrochloride, carboplatin, daptomycin, epinephrine, levetiracetam, oxaliplatin, paclitaxel, pantoprazole sodium, treprostinil, vasopressin, voriconazole, zoledronic acid, mometasone, fluticasone, ciclesonide, budesonide, beclomethasone, vilanterol, salmeterol, formoterol, umeclidinium, glycopyrrolate, tiotropium, aclidinium, indacaterol, salmeterol, and olodaterol.

As mentioned above, any of a variety of drugs can be delivered using a drug administration device. Examples of drugs that can be delivered using a drug administration device as described herein include Remicade® (infliximab), Stelara® (ustekinumab), Simponi® (golimumab), Simponi Aria® (golimumab), Darzalex® (daratumumab), Tremfya® (guselkumab), Eprex® (epoetin alfa), Risperdal Constra® (risperidone), Invega Sustenna® (paliperidone palmitate), Spravato® (esketamine), ketamine, and Invega Trinza® (paliperidone palmitate).

### Drug Housing

As described above, a dosage form can be provided in a holder that is appropriate for the particular dosage form being utilized. For example, a drug in a liquid dosage form can be held prior to administration within a holder in the form of a vial with a stopper, or a syringe with a plunger. A drug in solid or powder dosage form, e.g., as tablets, may be contained in a housing which is arranged to hold the tablets securely prior to administration.

The housing may comprise one or a plurality of drug holders, where each holder contains a dosage form, e.g., the drug can be in a tablet dosage form and the housing can be in the form of a blister pack, where a tablet is held within each of a plurality of holders. The holders being in the form of recesses in the blister pack.

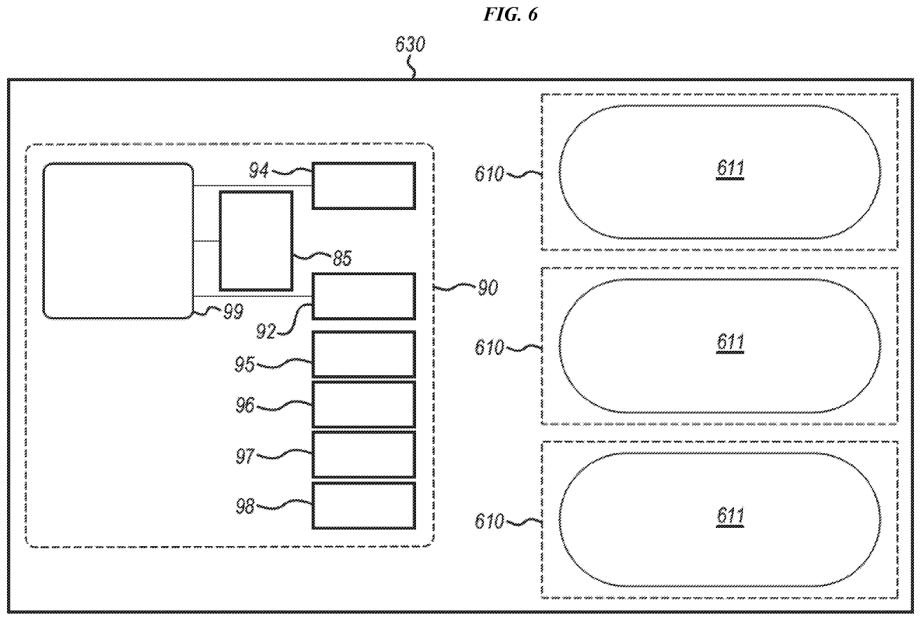

FIG. 6 depicts a housing 630 that comprises a plurality of drug holders 610 that each contain a dosage form 611. The housing 630 may have at least one environment sensor 94, which is configured to sense information relating to the environment in which the housing 630 is present, such as the temperature of the environment, time or location. The housing 630 may include at least one device sensor 92, which is configured to sense information relating to the drug of the dosage form 611 contained within the holder 610. There may be a dedicated location sensor 98 which is configured to determine the geographical location of the housing 630, e.g., via satellite position determination, such as GPS.

The housing 630 may include an indicator 85 which is configured to present information about the status of the drug of the dosage form 611 contained within the holder 610 to a user of the drug housing. The housing 630 may also include a communications interface 99 which can communicate information externally via a wired or wireless transfer of data pertaining to the drug housing 630, environment, time or location and/or the drug itself.

If required, the housing 630 may comprise a power supply 95 for delivering electrical power to one or more electrical components of the housing 630. The power supply 95 can be a source of power which is integral to housing 630 and/or a mechanism for connecting the housing 630 to an external source of power. The housing 630 may also include a device computer system 90 including processor 96 and memory 97 powered by the power supply 95 and in communication with each other, and optionally with other electrical and control components of the housing 630, such as the environment sensor 94, location sensor 98, device sensor 92, communications interface 99, and/or indicator 85. The processor 96 is configured to obtain data acquired from the environment sensor 94, device sensor 92, communications interface 99, location sensor 98, and/or user interface 80 and process it to provide data output, for example to indicator 85 and/or to communications interface 99.

The housing 630 can be in the form of packaging. Alternatively, additional packaging may be present to contain and surround the housing 630.

The holder 610 or the additional packaging may themselves comprise one or more of the device sensor 92, the environment sensor 94, the indicator 85, the communications interface 99, the power supply 95, location sensor 98, and device computer system including the processor 96 and the memory 97, as described above.

### Electronic Communication

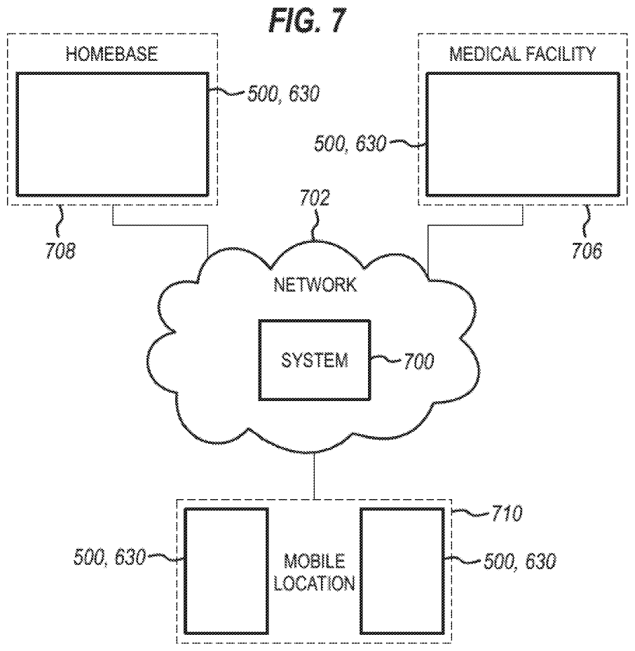

As mentioned above, communications interface 99 may be associated with the drug administration device 500 or drug housing 630, by being included within or on the housing 30, 630, or alternatively within or on the packaging 35. Such a communications interface 99 can be configured to communicate with a remote computer system, such as central computer system 700 shown in FIG. 7. As shown in FIG. 7, the communications interface 99 associated with drug administration device 500 or housing 630 is configured to communicate with a central computer system 700 through a communications network 702 from any number of locations such as a medical facility 706, e.g., a hospital or other medical care center, a home base 708 (e.g., a patient's home or office or a care taker's home or office), or a mobile location 710. The communications interface 99 can be configured to access the system 700 through a wired and/or wireless connection to the network 702. In an exemplary embodiment, the communications interface 99 of FIG. 6 is configured to access the system 700 wirelessly, e.g., through Wi-Fi connection(s), which can facilitate accessibility of the system 700 from almost any location in the world.

A person skilled in the art will appreciate that the system 700 can include security features such that the aspects of the system 700 available to any particular user can be determined based on, e.g., the identity of the user and/or the location from which the user is accessing the system. To that end, each user can have a unique username, password, biometric data, and/or other security credentials to facilitate access to the system 700. The received security parameter information can be checked against a database of authorized users to determine whether the user is authorized and to what extent the user is permitted to interact with the system, view information stored in the system, and so forth.

### Computer System

As discussed herein, one or more aspects or features of the subject matter described herein, for example components of the central computer system 700, processor 96, power supply 95, memory 97, communications interface 99, user interface 80, device indicators 85, device sensors 92, environment sensors 94 and location sensors 98, can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computer system may include clients and servers. A client and server are generally remote from each other and typically interact through a communications network, e.g., the Internet, a wireless wide area network, a local area network, a wide area network, or a wired network. The relationship of client and server arises by virtue of computer programs running on the respective computers and having a client-server relationship to each other.

The computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein, for example user interface 80 (which can be integrated or separate to the administration device 500 or housing 630), can be implemented on a computer having a display screen, such as for example a cathode ray tube (CRT) or a liquid crystal display (LCD) or a light emitting diode (LED) monitor for displaying information to the user. The display screen can allow input thereto directly (e.g., as a touch screen) or indirectly (e.g., via an input device such as a keypad or voice recognition hardware and software). Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. As described above, this feedback may be provided via one or more device indicators 85 in addition to the user interface 80. The device indicators 85 can interact with one or more of device sensor(s) 92, environment sensor(s) 94 and/or location sensor(s) 98 in order to provide this feedback, or to receive input from the user.

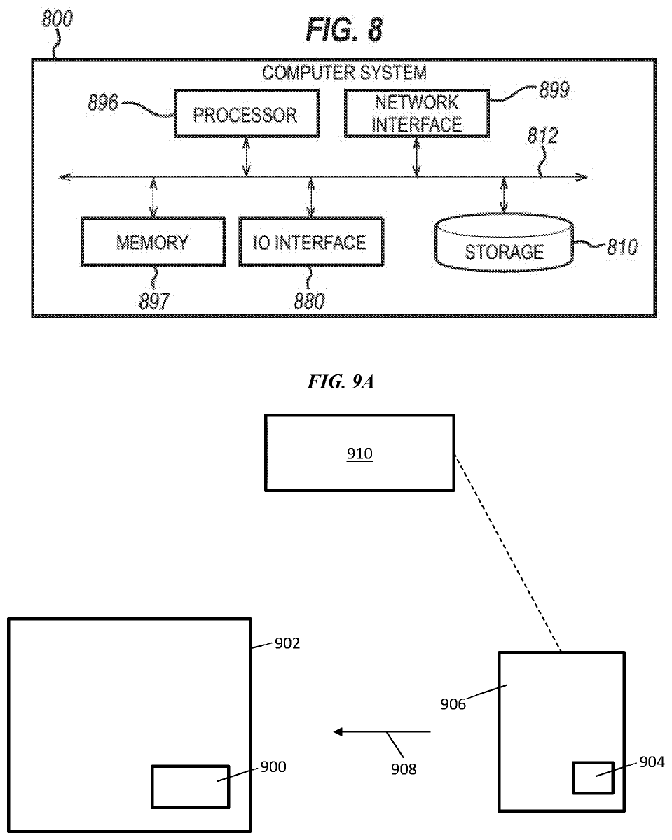

FIG. 8 illustrates one exemplary embodiment of the computer system 700, depicted as computer system 800. The computer system includes one or more processors 896 configured to control the operation of the computer system 800. The processor(s) 896 can include any type of microprocessor or central processing unit (CPU), including programmable general-purpose or special-purpose microprocessors and/or any one of a variety of proprietary or commercially available single or multi-processor systems. The computer system 800 also includes one or more memories 897 configured to provide temporary storage for code to be executed by the processor(s) 896 or for data acquired from one or more users, storage devices, and/or databases. The memory 897 can include read-only memory (ROM), flash memory, one or more varieties of random access memory (RAM) (e.g., static RAM (SRAM), dynamic RAM (DRAM), or synchronous DRAM (SDRAM)), and/or a combination of memory technologies.

The various elements of the computer system are coupled to a bus system 812. The illustrated bus system 812 is an abstraction that represents any one or more separate physical busses, communication lines/interfaces, and/or multi-drop or point-to-point connections, connected by appropriate bridges, adapters, and/or controllers. The computer system 800 also includes one or more network interface(s) 899 (also referred to herein as a communications interface), one or more input/output (IO) interface(s) 880, and one or more storage device(s) 810.

The communications interface(s) 899 are configured to enable the computer system to communicate with remote devices, e.g., other computer systems and/or devices 500 or housings 630, over a network, and can be, for example, remote desktop connection interfaces, Ethernet adapters, and/or other local area network (LAN) adapters. The IO interface(s) 880 include one or more interface components to connect the computer system 800 with other electronic equipment. For example, the IO interface(s) 880 can include high speed data ports, such as universal serial bus (USB) ports, 1394 ports, Wi-Fi, Bluetooth, etc. Additionally, the computer system can be accessible to a human user, and thus the IO interface(s) 880 can include displays, speakers, keyboards, pointing devices, and/or various other video, audio, or alphanumeric interfaces. The storage device(s) 810 include any conventional medium for storing data in a non-volatile and/or non-transient manner. The storage device(s) 810 are thus configured to hold data and/or instructions in a persistent state in which the value(s) are retained despite interruption of power to the computer system. The storage device(s) 810 can include one or more hard disk drives, flash drives, USB drives, optical drives, various media cards, diskettes, compact discs, and/or any combination thereof and can be directly connected to the computer system or remotely connected thereto, such as over a network. In an exemplary embodiment, the storage device(s) 810 include a tangible or non-transitory computer readable medium configured to store data, e.g., a hard disk drive, a flash drive, a USB drive, an optical drive, a media card, a diskette, or a compact disc.

The elements illustrated in FIG. 8 can be some or all of the elements of a single physical machine. In addition, not all of the illustrated elements need to be located on or in the same physical machine.

The computer system 800 can include a web browser for retrieving web pages or other markup language streams, presenting those pages and/or streams (visually, aurally, or otherwise), executing scripts, controls and other code on those pages/streams, accepting user input with respect to those pages/streams (e.g., for purposes of completing input fields), issuing HyperText Transfer Protocol (HTTP) requests with respect to those pages/streams or otherwise (e.g., for submitting to a server information from the completed input fields), and so forth. The web pages or other markup language can be in HyperText Markup Language (HTML) or other conventional forms, including embedded Extensible Markup Language (XML), scripts, controls, and so forth. The computer system 800 can also include a web server for generating and/or delivering the web pages to client computer systems.

As shown in FIG. 7, the computer system 800 of FIG. 8 as described above may form the components of the central computer system 700 which is in communication with one or more of the device computer systems 90 of the one or more individual drug administration devices 500 or housings 630. Data, such as operational data of the devices 500 or housings 630, medical data acquired of patients by such devices 500 or housings 630 can be exchanged between the central and device computer systems 700, 90.

As mentioned the computer system 800 as described above may also form the components of a device computer system 90 which is integrated into or in close proximity to the drug administration device 500 or housing 630. In this regard, the one or more processors 896 correspond to the processor 96, the network interface 799 corresponds to the communications interface 99, the IO interface 880 corresponds to the user interface 80, and the memory 897 corresponds to the memory 97. Moreover, the additional storage 810 may also be present in device computer system 90.

In an exemplary embodiment, the computer system 800 can form the device computer system 90 as a single unit, e.g., contained within a single drug administration device housing 30, contained within a single package 35 for one or more drug administration devices 500, or a housing 630 that comprises a plurality of drug holders 610. The computer system 800 can form the central computer system 700 as a single unit, as a single server, or as a single tower.

The single unit can be modular such that various aspects thereof can be swapped in and out as needed for, e.g., upgrade, replacement, maintenance, etc., without interrupting functionality of any other aspects of the system. The single unit can thus also be scalable with the ability to be added to as additional modules and/or additional functionality of existing modules are desired and/or improved upon.

The computer system can also include any of a variety of other software and/or hardware components, including by way of example, operating systems and database management systems. Although an exemplary computer system is depicted and described herein, it will be appreciated that this is for sake of generality and convenience. In other embodiments, the computer system may differ in architecture and operation from that shown and described here. For example, the memory 897 and storage device 810 can be integrated together or the communications interface 899 can be omitted if communication with another computer system is not necessary.

### Implementations

Drug delivery systems and methods for monitoring the exposure of a drug to one or more exposure conditions, such as an environmental condition, are provided. These systems and methods allow for the monitoring and/or tracking of exposure, e.g., intensity level(s) and/or duration(s), of a drug to one or more exposure conditions that can affect performance of the drug, e.g., viability, longevity, and potency. Viability of a drug generally refers to efficacy of the drug, e.g., the drug's ability to produce a particular effect. Longevity of a drug generally refers to a length of time the drug can produce a particular effect. Potency of a drug generally refers to an amount of the drug needed to produce a particular effect. The monitoring or tracking of the drug from the point of manufacture to administration, or a portion thereof, can allow for early identification of non-viable drugs, as well as modification of drug dosage and/or shelf-life based upon the exposure monitoring or tracking. Thus, the present systems and methods may reduce the risk of administering a drug at a dosage that has been rendered ineffective due to exposure conditions, and may reduce the risk of non-viable drugs being administered to patients.

As mentioned above, any of a variety of drugs can be delivered using a drug administration device. Examples of drugs that can be delivered using a drug administration device as described herein include Remicade® (infliximab), Stelara® (ustekinumab), Simponi® (golimumab), Simponi Aria® (golimumab), Darzalex® (daratumumab), Tremfya® (guselkumab), Eprex® (epoetin alfa), Risperdal Constra® (risperidone), Invega Sustenna® (paliperidone palmitate), Spravato® (esketamine), ketamine, and Invega Trinza® (paliperidone palmitate).

In general, drug delivery systems and methods described herein include active or passive sensing mechanisms that can monitor at least one exposure condition of a drug. In some instances, the active or passive sensing mechanisms can also track the extent of the drug's exposure (e.g., frequency, intensity, and/or duration). As a result, the information related to the exposure condition itself and/or the extent of exposure can be used to determine the viability and efficacy of the drug prior to administration and/or prior to distribution of the drug in commerce.

The drug delivery systems described herein can include one or more drug administration devices, such as any one or more of the drug administration devices discussed above. For example, the one or more drug administration devices can include at least one drug holder having a drug disposed therein. The one or more drug administration devices can be configured to deliver a drug to a subject. For example, the one or more drug administration devices can include a drug dispensing mechanism that is configured to deliver at least a portion of the drug to a user upon activation, e.g., upon actuation of a drug delivery actuator by a user. Further, the one or more drug administration devices can include a housing that is configured to accommodate the drug holder and dispensing mechanism. Suitable drug administration devices and features thereof are discussed in more detail above.

Further, the drug delivery systems described herein can also include at least one sensor that can be configured to monitor or detect at least one exposure condition of a drug. Examples of exposure conditions include geographic location (e.g., as sensed by a location sensor configured to sense GPS or other location), time (e.g., as sensed by a timer or a clock device such as an atomic clock), date (e.g., as sensed by a timer), temperature (e.g., as sensed by a temperature sensor), ultraviolet (UV) exposure (e.g., as sensed by a UV sensor configured to sense UV level), pH (e.g., as sensed by a pH sensor configured to sense pH level), and humidity (e.g., as sensed by a humidity sensor configured to sense humidity level). Alternatively, or in addition, the at least one sensor can be configured to track the frequency, duration, and/or intensity of an adverse exposure event experienced by the drug prior to administration, e.g., a temperature spike during transport or storage of the drug as sensed by a temperature sensor configured to sense temperature and a timer configured to provide time stamp data for the sensed temperature data. In various embodiments, a sensor includes an image capturing device such as a camera, and a processor is configured to analyze image(s) and/or video(s) captured by the image capturing device, such as to analyze any food intake and/or patient skin reaction to the drug. U.S. Patent Pub. No. 2012/0330684 entitled “Medication Verification And Dispensing” published Dec. 27, 2012, which is incorporated by reference herein in its entirety, further describes image capturing devices. U.S. Patent Pub. No. 2002/0014951 entitled “Remote Control For A Hospital Bed” published Feb. 7, 2002, and U.S. Patent Pub. No. 2007/0251835 entitled “Subnetwork Synchronization And Variable Transmit Synchronization Techniques For A Wireless Medical Device Network” published Nov. 1, 2007, further discuss various sensors and are incorporated by reference herein in their entireties.

### Active Sensing Mechanisms

A. Shelf-Life and Supply Chain Monitoring Systems

In some embodiments, the drug delivery systems can include two sensors. The first sensor can be associated with the drug administration device and/or a packaging unit for the drug administration device, and the second sensor can be associated with the drug itself. The packaging unit can contain one or more drug administration devices.

As discussed in more detail below, the first and second sensors can be used to monitor exposure conditions of the drug prior to the drug being administered to a patient. This may help ensure that at the time of administration, the drug is viable and being delivered at an effective dosage. Moreover, this monitoring may also aid in detection of non-viable drugs early on in the supply chain and/or between intermittent dosing. As a result, drug manufacturers can recall non-viable drugs at an early stage, e.g., prior to packaging and/or distribution, which may lead to decreased recall costs and avoid the potential health risks to the patients.

The first sensor can be configured to monitor at least one exposure condition of the drug while the drug is disposed within a drug administration device. Alternatively, or in addition, the first sensor can be configured to monitor at least one exposure condition of the drug while the drug administration device is within the packaging unit. As such, the first sensor can be configured to monitor at least one exposure condition of the drug after the drug is associated with the drug administration device. As a result, the first sensor can function as a shelf-life monitor for the drug once the drug is disposed within the device.

The second sensor can be configured to monitor at least one exposure condition of the drug from an initial time before the drug is associated with the drug administration device to a second time in which the drug is associated with the drug administration device and the first sensor is activated. For example, the second sensor can be configured to monitor at least one exposure condition of the drug through the entire drug supply chain process, or alternatively, during different stages thereof. In general, a drug's supply chain begins at manufacturing of the drug and proceeds in order to packaging of the drug, storage of the drug in its packaging, and distribution of the drug in its packaging. In one embodiment, the initial time is the time that the drug enters the supply chain, e.g., when the drug itself is manufactured.

The data acquired by the first sensor and/or second sensor can be communicated to a processor through a communications interface. The communications interface can be associated with the drug administration device or drug holder, or alternatively within or on the packaging unit for the drug administration device, as discussed above. The processor can be remote from or local to the drug administration device. Further, the processor can be a component of a computer system, such as computer system 700, 800 shown in FIGS. 7 and 8. In use, once the data is received by the processor, the processor can process the data and provide a data output. In one example, the data output can be an expiration date, which can be determined by taking into account the data acquired by the first and/or second sensors. For example, the processor can be configured to determine the expiration date by determining an elapsed amount of time after the drug has been associated with the drug administration device as indicated by the first sensor (or other sensor) or an elapsed amount of time after the drug itself is manufactured as indicated by the second sensor (or other sensor). The processor can also be configured to compare the determined elapsed amount of time with the drug's predetermined expiration date as set by the manufacturer (or other quality controller) to determine whether the expiration date has passed. The processor can also be configured to adjust the elapsed amount of time based on the data acquired by the first and/or second sensors to account for intensity and duration of any exposure condition of the drug since the drug's association with the drug administration device (first sensor data) and/or since the drug's manufacture (second sensor data). The processor can be configured to access a lookup table that is stored in a memory and that stored predetermined metrics for the drug. The predetermined metrics can associate the drug with each of one or more exposure conditions and indicate the exposure condition's effect on the drug's expiration date, e.g., by indicating how much time the drug's expiration date should be adjusted downward (if at all) for particular time durations of the exposure condition.

In some embodiments, the expiration date can be for a batch of the drug, for example, when the drug has yet to be disposed within the drug administration device. Alternatively or in addition, the expiration date can be for the drug disposed in the drug administration device. Further, the processor can also be configured to provide a data output to the drug administration device indicating that the batch of the drug and/or the drug in the device itself is beyond its expiration date. For example, the data output can be in the form of a warning. Alternatively, or in addition, upon receiving the data output, the drug administration device can be configured to prevent drug delivery.

A warning as discussed herein can be to a user of the drug administration device and/or to a third party (e.g., a manufacturer of the drug, a pharmacy that provided the drug by prescription, a cloud service configured to communicate with pharmacies, a health care provider (HCP) of the patient prescribed the drug, etc.). Providing a warning to the user may help prevent the drug from being delivered from the drug administration device and thus help avoid adverse patient effects and/or allow the user to obtain new drug before a next dosage is due. Providing a warning to the third party as a cloud service may (1) facilitate automatic product replacement by allowing the cloud service to automatically reorder the drug and/or drug administration device loaded with the drug from the user's pharmacy, (2) allow the cloud service to automatically generate a complaint report that is transmitted from the cloud service to another third party, e.g., a manufacturer of the drug, a pharmacy that provided the drug by prescription, a health care provider that prescribed the drug, etc., that the other third party may use to evaluate their business, take remedial action, etc., (3) allow the cloud service to automatically generate a request to a quality control unit, such as a quality control team at the drug's manufacturer, for consultation of what step(s) the user, the user's HCP, the drug's manufacturer, and/or another party should take, and/or (4) associate the particular drug administration device (e.g., as identified with a product identification code included in the warning) with a serialization that can be traced to a specific distribution leg in the supply chain, should the excursion happen with the user then the drug administration device may not be refundable or replaced due to a history of known user error and/or the user can be reminded of appropriate storage conditions for the drug administration device (e.g., message shown on a display of the drug administration device, email sent to the user associated with the drug administration device, the patient's HCP informed of the user errors for discussion with the patient, etc.). Providing a warning to a HCP of the patient prescribed the drug, e.g., a warning indicative of a missed dose or a delayed dose, may allow the HCP to have a more accurate history of the patient's medication usage for use in evaluating the patient's treatment.

Another example of the data output of the processor after the processor processes the data is an excursion condition state, which can be determined by taking into account the data acquired by the first and/or second sensors. For example, the processor can be configured to compare data received from the first sensor and/or second sensor with a predetermined threshold or range indicative of a safe environmental condition. If the received data is outside of the predetermined safe range, above the predetermined safe threshold, or below the predetermined safe threshold as appropriate for the particular environmental condition, the data output can be in the form of a warning indicating that the drug has experienced at least one environmental condition during its life so far in the supply chain that its performance has been adversely affected enough such that the drug should not be delivered from the drug administration device.

The second sensor can also be configured to track different stages of the supply chain and the duration of each stage. Rushes or delays in the supply chain can also have an impact on the drug. For example, production or storage delays of the drug itself can negatively affect the shelf-life of the drug before the drug is disposed within the drug administration device. As such, in some embodiments, the second sensor can be configured to control the activation of the first sensor so as to prevent premature activation that can occur when the drug encounters unanticipated temporal events (rushes or delays) between the time of drug manufacture to the time the drug is associated with the drug administration device. In this way, the activation of the first sensor can be tailored in response to temporal events in the supply chain. For example, the second sensor can transmit data to a processor through a communications interface, as discussed above, and the processor can be configured to provide a data output to the first sensor that delays or expedites activation of the first sensor.

Various embodiments of sensors and sensor communication are further described in U.S. Patent Pub. No. 2007/0251835 entitled “Subnetwork Synchronization And Variable Transmit Synchronization Techniques For A Wireless Medical Device Network” published Nov. 1, 2007, which is incorporated by reference herein in its entirety.

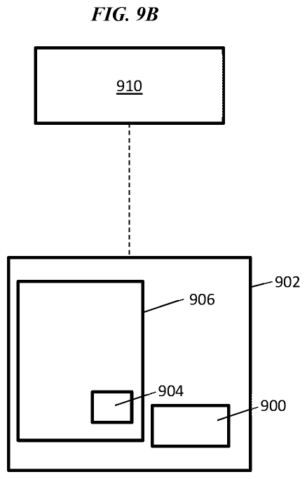

FIG. 9A is a block schematic showing a first sensor 900 associated with a drug administration device 902 and a second sensor 904 associated with a drug holder 906 that is configured to be disposed within the drug administration device 902. While not shown, the drug holder 906 includes drug disposed therein. As a result, the second sensor 904 can be configured to monitor at least one exposure condition of the drug prior to the drug being associated with the drug administration device 902 and the first sensor 900 being activated, as shown in FIG. 9A. Further, in this illustrated embodiment, the second sensor 904 is in communication with a remote processor 910 such that the sensed data of the second sensor 904 can be transmitted thereto, as discussed above. Moreover, as shown, the drug is still within a supply chain 908, which can begin at the time of manufacturing, until the drug is associated within the drug administration device 902 (FIG. 9B).

Once the drug holder 906 is placed within the drug administration device 902, as shown in FIG. 9B, the drug is associated with the drug administration device 902, and the first sensor 900 can be activated (e.g., by the second sensor 904). As a result, the first sensor 900 can be configured to monitor at least one exposure condition of the drug after the drug is associated with the drug administration device 902. Further, in this illustrated embodiment, the first sensor 900 is in communication with the remote processor 910 such that the sensed data of the first sensor 900 can be transmitted thereto, as discussed above. As a result, the sensed data from the first and second sensors 900, 904 can be used to monitor the drug from the time of manufacturing to the time of administration.

In certain embodiments, the first and/or second sensors can have an independent exposure and shelf life. For example, the sensitivity of the first and/or second sensors can be affected over time by exposure to conditions, some of which can be representative of those experienced by the drug disposed within the drug administration device. This can ultimately lead to deactivation of the drug administration device, thereby preventing the drug administration device from delivering drug until the compromised sensor is replaced. As such, in certain embodiments, prior to deactivation, the drug administration device can be configured to provide at least one cue indicating that the first sensor and/or second sensor has been compromised. In this way, a user can be afforded enough time to retrieve a new array, e.g., test strips, chemical assay, etc., and therefore avoid any interruption in treatment that would otherwise occur during device use.

B. Detecting and Tracking Systems

In some embodiments, the drug delivery systems include a housing, a sensor, and a communications interface. The housing contains one or more drug administration devices having a drug disposed therein. For example, each of the one or more drug administration devices can be configured to receive at least one drug holder having a drug disposed therein. In certain embodiments, the housing can be a packaging unit for the one or more administration devices.

The sensor can be associated with the housing, the one or more drug administration devices, and/or at least one drug holder. The sensor can be configured to detect at least one exposure condition, such as an environmental condition, of the drug. The sensor can also be configured to sense the intensity and/or duration of the at least one exposure condition. For example, the sensor can be configured to detect at least one exposure condition that the drug experiences from the point of manufacture to the point of administration, or any period of time therebetween. The sensed data representative of the at least one exposure condition of the drug can ultimately be used as a basis for adjusting drug dosing to account for reduced performance of the drug due to the at least one exposure condition, adjusting dose delivery rate (e.g., rate of injection, etc.) to account for the at least one exposure condition, and/or determining drug viability. For example, the at least one exposure condition may have reduced potency of the drug such that increased dosage is warranted. For another example, the at least one exposure condition may indicate that a temperature of the drug is below a predetermined threshold temperature indicative of patient comfort such that a slower rate of injection would be more comfortable for the patient as the drug warms. The sensed data can be transmitted, e.g., through a communications interface, to a processor that is configured to analyze and determine the effect of the at least one exposure condition on the drug and/or how long the patient should wait before room temperature may bring the drug to a more comfortable temperature given the sensed current temperature. The sensed data can be transmitted to the processor at a regular sampling rate, on demand, or continuously.

In some embodiments, the sensor can be associated with the housing for detecting and tracking exposure condition(s) of the drug during shipment and/or storage. For example, the sensor can be disposed on or within the housing and be configured to measure and log exposure condition(s) that can affect drug performance. In this way, more precise and continuous data gathering can be obtained during bulk transport. Alternatively, or in addition to, the sensor can be associated with the one or more drug administration devices. The sensor can transmit the data representative of the exposure condition(s) to a processor, e.g., through a communications interface, that is configured to compute and transmit output data that can be used to identify compromised drug shipments. As a result, compromised drug shipments can be more accurately detected. That is, the output data can be used to make more informed decisions as to whether the compromised drugs should be saved or discarded before such drug reaches any patient. Further, the output data can be used to determine whether certain modifications need to be made to ensure effective drug delivery to a patient (e.g., adjustment of a dosage of the drug, adjustment of a rate of delivery of the drug from the drug administration device, etc.).

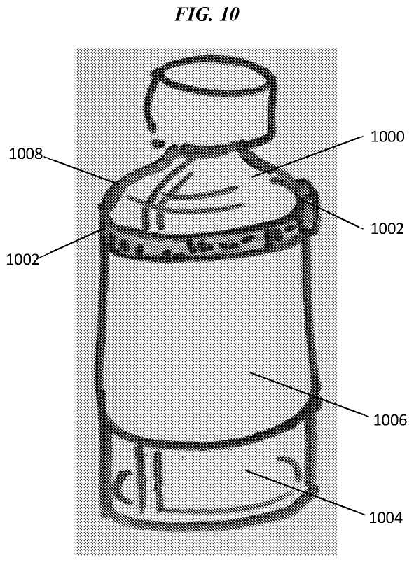

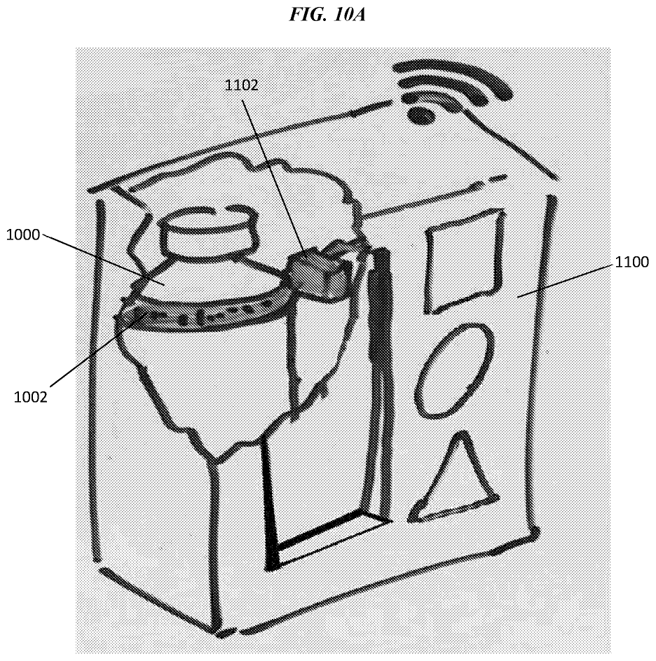

In other embodiments, the sensor can be associated with a drug holder that is configured to be disposed within a drug administration device. FIG. 10 illustrates an exemplary drug holder 1000 having a sensor 1002 associated therewith. While the drug holder 1000 can have variety of configurations, in this illustrated embodiment, the drug holder 1000 includes a body 1004 defining a reservoir chamber configured to hold a drug (not shown). In other embodiments, the drug holder 1000 can have other configurations, shapes, and sizes.

Further, as shown in FIG. 10, a label 1006 is disposed about a portion of an outer surface 1008 of the drug holder 1000. As shown, in this example the sensor 1002 is disposed on a portion of the label 1006. The sensor 1002 is configured to track at least one exposure condition of a drug (not shown) disposed within the drug holder 1000. For example, the sensor 1002 can be configured to track temperature and/or ultraviolet exposure throughout a time period, e.g., from the time the drug is disposed within the drug holder 1000 to the time of administration, or any portion therebetween. Further, the sensor 1002 can be configured to log or store the tracking data. In certain embodiments, the sensor 1002 can also be configured to track the expiration date of the drug.

Any data tracked by a sensor associated with a drug holder can be communicated to a drug administration device. For example, as shown in FIG. 10A, a drug administration device 1100 can include an electrical contact 1102 that is configured to read the sensed data from a sensor 1002 on a drug holder 1000. While the drug administration device 1100 can have variety of configurations, such as those discussed above, the drug administration device 1100, as shown in FIG. 10A, is an exemplary infusion pump. As shown in FIG. 10A, the drug holder 1000 is housed within the drug administration device 1100 and the sensor 1002 is positioned in close proximity to the electrical contact 1102. As such, once the sensor 1002 is positioned close to or in direct contact with the electrical contact 1102, the sensor 1002 is read by the electrical contact 1102 (e.g., a reader) and the data from the sensor 1002 is transmitted to a remote processor, such as processor 96, 896 shown in FIGS. 5B and 8.

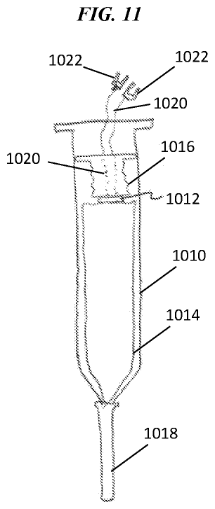

FIG. 11 illustrates another exemplary drug holder 1010 having a sensor 1012 associated therewith. As mentioned above, a drug holder can have variety of configurations, but in this illustrated embodiment, the drug holder 1010 includes a syringe defining a reservoir chamber 1014 configured to hold a drug (not shown). The drug holder 1010 includes a plunger 1016 configured to be pushed and thereby cause the drug to be ejected from the syringe's needle (obscured by a needle cover 1018 in FIG. 11). In this embodiment, the sensor 1012 is disposed on a bottom of the plunger 1016, e.g., a side of the plunger 1016 that is nearest the reservoir chamber 1014 and is configured to contact the drug within the reservoir chamber 1014. The sensor 1012 is thus configured to contact the drug within the reservoir chamber 1014. The sensor 1012 is also configured to track at least one exposure condition, e.g., temperature, UV, pH, pressure, etc., of the drug disposed within the drug holder 1010, similar to that discussed above regarding the sensor 1002 of FIGS. 10 and 10A.

Any data tracked by the sensor 1012 can be communicated to a drug administration device that houses the drug holder 1010. A lead 1020 extends from the sensor 1012 and has a electrical contact 1022 at its end opposite the sensor 1012. The electrical contact 1022 is in the form of a connector in this illustrated embodiment and is configured to connect to a corresponding connector of the drug administration device to electrically connect the sensor 1012 to at least one electrical component of the drug administration device to facilitate transmission of data monitored by the sensor 1012, using a communications interface of the drug administration device, to a remote processor, such as processor 96, 896 shown in FIGS. 5B and 8. The lead 1020 extends through the plunger 1016, which may help protect the lead 1020 from damage. The lead 1020 in this illustrated embodiment includes two leads 1020, but another number of leads (and associated connectors) may be used.

In the illustrated embodiments of FIGS. 10-11, the data is wirelessly transmitted to a remote processor relative to the drug administration device through a communications interface, such as communications interface 99, 899 shown in FIGS. 5B and 8. In other embodiments, the data is transmitted to the remote processor through a wired connection. Alternatively, the data can be transmitted to local processor, e.g., a processor located on or within the drug administration device. As discussed in more detail below, the processor can compare the data against defined criteria and determine whether the data satisfies the criteria. In instances where the data does not satisfy the criteria, the processor can provide data output to the drug administration device that can modify the dosage of the drug or prevent administration of the drug altogether.

In certain embodiments, the processor is a component of a computer system, such as computer system 700, 800 shown in FIGS. 7 and 8, which can also include memory. As such, the sensor and the processor can be part of a closed-loop feedback system. The stored data within the memory can include predetermined threshold(s) for one or more exposure conditions of the drug. During data sensing, the processor can receive feedback input from the sensor. The processor can aggregate the received feedback input(s), perform any necessary calculations, compare it to the predetermined threshold for the corresponding exposure condition, and provide data output. If at any time during an exposure condition, the processor determines that the received feedback exceeds a predetermined control threshold, the processor can modify the data output to adjust the dosage of the drug and/or a rate of delivery of the drug. Alternatively, or in addition, if at any time during an exposure condition, the processor determines that the received feedback input exceeds a maximum predetermined threshold or is less than a minimum predetermined threshold, the processor can modify the data output to prevent administration of the drug due to non-viability.

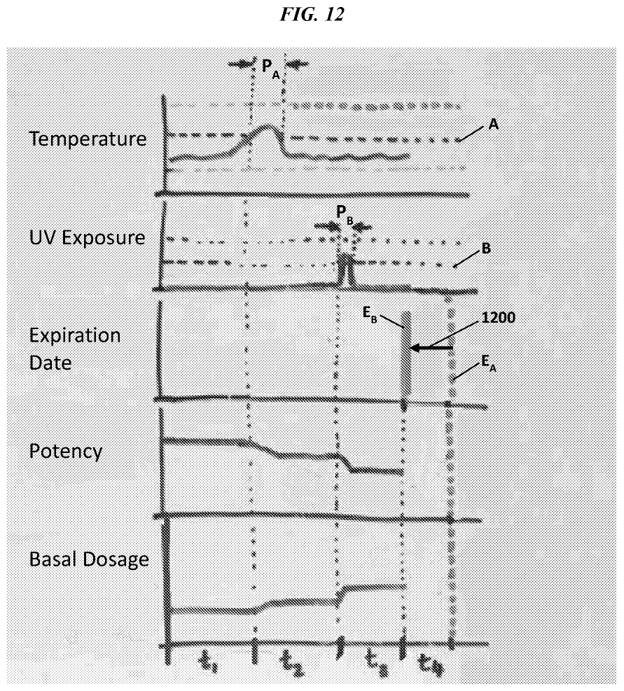

For example, in one embodiment, as shown in FIG. 12, a sensor, such as sensor 1002 shown in FIGS. 10 and 10A or the sensor 1012 shown in FIG. 11, can be configured to track temperature, ultraviolet exposure of a drug over four different time intervals T₁, T₂, T₃, T₄. A person skilled in the art will appreciate, however, that the following discussion is also applicable to other exposure conditions, e.g., humidity, pressure, pH, etc.

In this exemplary embodiment, if the processor determines that the drug is being exposed to a temperature that exceeds a predetermined temperature control threshold (A) during any time interval, the processor can be configured to cause an increase of the basal dosage of the drug in response to the decrease in potency. For example, the processor can transmit a data output characterizing the adjusted dosage to the drug administration device associated with the drug. In this illustrated embodiment, the processor determined that the exposure temperature of the drug exceeded the predetermined temperature control threshold A for a period of time, P_{A}, during the second time interval T₂. As shown, this increase in temperature for a period of time P_{A} caused the potency of the drug to decrease. This is because the potency of the drug is a function of the intensity and duration of an exceeding exposure event. Further, the potency of the drug is also a function of the frequency of the exceeding exposure event.

Similarly, in this exemplary embodiment, if the processor determines that the drug is being exposed to UV that exceeds a predetermined UV control threshold, B, the processor can be configured to increase the basal dosage of the drug in response to the decrease in potency. In this illustrated embodiment, the processor determined that the UV exposure of the drug exceeded the predetermined control threshold B for a period of time, P_{B}, during the third time interval T₃. As shown, this increase in UV for a period of time P_{B} caused the potency of the drug to further decrease.

Further, if a drug is exposed to an adverse exposure event, the drug's shelf life can be affected. For example, as shown in FIG. 12, since the temperature and UV exposure exceeded the predetermined temperature control threshold and predetermined UV control threshold, respectively, the drug's shelf life was decreased, as denoted by arrow 1200. In particular, the shelf-life decreased from E_{A} to E_{B}. This resulted in a loss of drug viability over the fourth time interval T₄. As such, the expiration date of the drug was expedited due to the exceeding temperature and UV exposure conditions experienced by the drug. Thus, the expiration date of the drug can be a function of the intensity and duration of any exposure condition of the drug. A person skilled in the art will therefore appreciate that in other instances, the drug's shelf-life can be increased.

In some embodiments, the drug delivery systems can include a device indicator, such as device indicator 85 shown in FIG. 5B. The indicator can be associated with the drug holder and/or the drug administration device. The indicator can be configured to communicate a condition of the drug to a user. For example, in one embodiment, the indicator can communicate an expiration date of the drug, a viability state of the drug, a potency of the drug, and/or a recommended dosage of the drug, and/or the like to a user.

### Passive Mechanisms

A. Electronic Confirmation Systems

In some embodiments, the drug delivery systems can include a drug status indicator and a reader that is configured to detect the drug status indicator. The drug status indicator can be configured to indicate an extent (e.g., frequency, intensity, and/or duration) of an exposure of the drug to at least one environmental condition (e.g., temperature, UV exposure, humidity, etc.). For example, the drug status indicator can be responsive to an intensity and/or duration of an environmental condition.

The drug status indicator can have a variety of configurations. For example, in one embodiment, the drug status indicator can include a color change material that can be detected by the reader, e.g., an image sensor configured to capture an image of the drug status indicator and provide the image to a processor for analysis, which can include comparison of the color of the drug status indicator in the image with previously captured image(s) of the drug status indicator to determine if a color change has occurred and/or a predetermined color designated as “normal” to determine if the current color of the drug status indicator deviates from normal. The color change material can be used as a measure of exposure of the drug to the at least environmental condition. That is, the color change material can be configured to change color when the drug is exposed to an adverse environmental condition for a sufficiently long period of time. In use, this color change can be detected by the reader.

In other embodiments, the drug status indicator can include a reactive agent that can be configured to interact with the drug, e.g., the drug status indicator can be added to a segmented portion of a housing such as by being integrated into a material of the housing. In use, if the drug is still viable, the interaction can create a specific color, fluorescence, and/or the like that can be detected by the reader, e.g., an image sensor configured to capture an image of the drug status indicator and provide the image to a processor for analysis, which can include comparison of the color, fluorescence, etc. of the drug status indicator in the image with previously captured image(s) of the drug status indicator and/or a predetermined color, fluorescence, etc. designated as “normal.” If the drug is non-viable, there is either no interaction or the resulting interaction creates a specific color, fluorescence, and/or the like that is undetectable by the reader.

In other embodiments, the drug status indicator can be a degradable element, e.g., a degradable circuit, that is impacted when exposed to an adverse environmental condition. That is, upon exposure to an environmental condition, the degradable element can degrade if the intensity and/or duration of the environmental condition exceeds a predetermined threshold. As such, the amount of degradation can be indicative of the condition of the drug, for example, at the time of administration. Further, this degradation can ultimately render the degradable element undetectable by the reader, e.g., an electrical circuit configured to communicate with the degradable circuit with ceasing of responses from the degradable circuit to requests from the reader being indicative of the degradable circuit having degraded or an image sensor configured to capture an image of the drug status indicator with the degradable element no longer being visible in the image being indicative of the degradable element having degraded, thereby indicating that the drug is non-viable. Thus, the degradable element can function as a switch such that the detection or non-detection thereof signifies that the drug is viable or non-viable, respectively.

The degradable element can have a variety of configurations. In one embodiment, the degradable element can include one or more bioabsorbable and biocompatible polymers, including homopolymers and copolymers, that are configured to detect humidity levels experienced by the drug as the degradable element degrades in the presence of water. Examples of homopolymers and copolymers include p-dioxanone (PDO or PDS), polyglycolic acid (PGA), poly(lactic-co-glycolic acid) (PLGA), polycaprolactone (PCL), trimethylene carbonate (TMC), and polylactic acid (PLA), poly(glycolic acid-co-lactic acid) (PLA/PGA) (e.g., PLA/PGA materials used in Vicryl, Vicryl Rapide, PolySorb, and Biofix), polyurethanes (such as Elastane, Biospan, Tecoflex, Bionate, and Pellethane fibers), polyorthoesters, polyanhydrides (e.g., Gliadel and Biodel polymers), polyoxaesters, polyesteramides, and tyrosine-based polyesteramides. Other examples of copolymers include poly(lactic acid-co-polycaprolactone) (PLA/PCL), poly(L-lactic acid-co-polycaprolactone) (PLLA/PCL), poly(glycolic acid-co-trimethylene carbonate) (PGA/TMC) (e.g., Maxon), Poly(glycolic acid-co-caprolactone) (PCL/PGA) (e.g., Monocryl and Capgly), PDS/PGA/TMC (e.g., Biosyn), PDS/PLA, PGA/PCL/TMC/PLA (e.g., Caprosyn), and LPLA/DLPLA (e.g., Optima), poly(L-lactic acid) (PLLA), polyethylene terephthalate (PET), polyhydroxyalkanoate (PHA), a copolymer of glycolide and ε-caprolactone (PGCL), a copolymer of glycolide and -trimethylene carbonate, poly(glycerol sebacate) (PGS), polyesters, polyoxaesters, polyetheresters, polycarbonates, polyamide esters, polyanhydrides, polysaccharides, poly(ester-amides), tyrosine-based polyarylates, polyamines, tyrosine-based polyiminocarbonates, tyrosine-based polycarbonates, poly(D,L-lactide-urethane), poly(hydroxybutyrate), poly(B-hydroxybutyrate), poly(E-caprolactone), polyethyleneglycol (PEG), poly[bis(carboxylatophenoxy)phosphazene]poly(amino acids), pseudo-poly(amino acids), absorbable polyurethanes, poly(phosphazine), polyphosphazenes, polyalkyleneoxides, polyacrylamides, polyhydroxyethylmethylacrylate, polyvinylpyrrolidone, polyvinyl alcohols, poly(caprolactone), polyacrylic acid, polyacetate, polypropylene, aliphatic polyesters, glycerols, copoly(ether-esters), polyalkylene oxalates, polyamides, poly(iminocarbonates), polyalkylene oxalates, and combinations thereof. As understood by a person skilled in the art, degradation can be measured by ionizing the one or more polymers, or alternatively, doping the one or more polymers with a conductive material, which can allow for a resistive measure when the degradable element is in intact (undamaged). As such, the degradation of the one or more polymers is proportional to the degradation of the resistive circuit. Alternatively, or in addition, the degradable element can be formed of one or more copolymers, e.g., poloxamers, of different viscosity and/or molecular weight. In this way a predictable degradation profile can be created.

In other embodiments, the drug status indicator can be configured to indicate that it is safe to begin mixing the drug. Some drugs need to be mixed or reconstituted prior to use, such as glucagon and other drugs. The drug administration device may thus be a device configured to allow drug mixing, such as a manual dual chamber syringe or a motorized dual chamber system that allows for the placement of a drug vial and a diluent vial. Such a drug administration device can be a reusable or disposable battery powered system. The drug status indicator can be configured to indicate that the drug is within a predetermined safe range for each of one or more environmental conditions (e.g., temperature and humidity) and is thus ready to begin mixing. For example, a temperature sensor can be configured to sense temperature and communicate sensed temperature data to a processor, a humidity sensor can be configured to sense humidity and communicate sensed humidity data to the processor, and the processor can be configured to determine whether the sensed temperature and sensed humidity are each within their respective predetermined safe ranges. If one or both of the sensed temperature and sensed humidity are not within their respective predetermined safe ranges, the processor can be configured to generate a warning to a user of the drug administration device to indicate that the drug is not in a condition to be mixed and/or to generate a warning to a third party to indicate that the drug is not in a condition to be mixed. If each of the sensed temperature and sensed humidity are within their respective predetermined safe ranges, the processor can be configured to take no action since the drug is in a proper state for mixing. The one or more environmental conditions can also be used as an indicator that the drug has been properly mixed, e.g., the one or more environmental conditions being used to determine whether the mixed drug is within a predetermined safe range for each of the environmental conditions.

The reader can be in wired or wireless communication with a processor. As such, when the reader detects or is unable to detect the drug status indicator, this information can be transmitted to the processor. The processor can be configured to prompt a cue to a user when the reader stops or is unable to detect the drug status indicator as expected for a viable drug, e.g., due to the degradation or color change of the drug status indicator in response being exposed to an environmental condition that exceeds a threshold exposure duration and/or a threshold exposure intensity.

In certain embodiments, the drug status indicator can be associated with a drug administration device. For example, the drug status indicator can be on or within the drug administration device. Alternatively, the drug status indicator can be on a housing for the drug administration device. The housing can be a packing unit for one or more drug administration devices. In one embodiment, the drug status indicator can be in the form of an electrochromic paste inserted on or within the drug administration device, or alternatively on or within the housing, that is configured to detect the exposure temperature during shipment and/or storage. In use, once the drug administration device or housing has reached a destination, the reader can be used to detect the drug administration device and determine whether temperature limits were maintained during shipment.

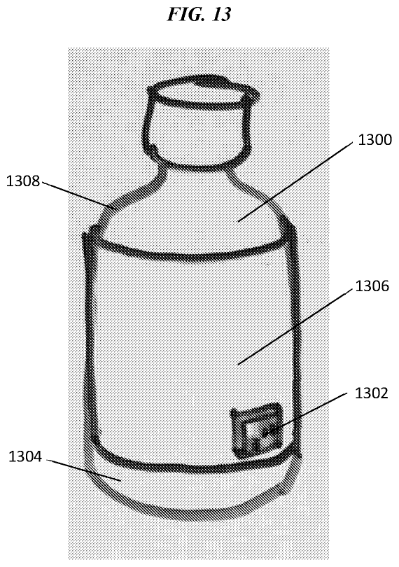

In other embodiments, the drug status indicator can be associated with a drug holder that is configured to be disposed within a drug administration device. FIG. 13 illustrates an exemplary drug holder 1300 having a drug status indicator 1302 associated therewith. While the drug holder 1300 can have a variety of configurations, the drug holder 1300 includes a body 1304 defining a reservoir chamber configured to hold drug (not shown). In other embodiments, the drug holder 1300 can have other configurations, shapes, and sizes.

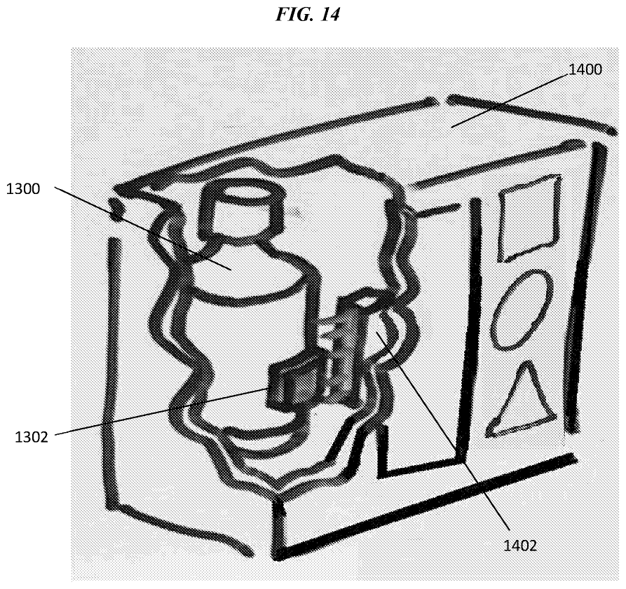

Further, as shown in FIG. 13, a label 1306 is disposed on an outer surface 1308 of the drug holder 1300. The label 1306 includes the drug status indicator 1302, which is configured to indicate an extent of exposure of the drug to at least one environmental condition. While the drug status indicator 1302 can have a variety of configurations, the drug status indicator 1302, as shown in FIG. 13, is a degradable circuit. Once the drug holder 1300 is disposed within a drug administration device 1400, as shown in FIG. 14, a reader 1402 within the drug administration device 1400 can be used to detect the degradable circuit 1302. While the drug administration device 1400 can have variety of configurations, such as those discussed above, the drug administration device 1400, as shown in FIG. 14, is an exemplary infusion pump. In this illustrated embodiment, if the degradable circuit 1302 has been degraded, the reader 1402 will be unable to detect it, thereby indicating that the drug has been exposed to a temperature, a humidity, or an amount of ultraviolet light that has adversely impacted the drug to the point of non-viability.

B. Visual Confirmation Systems

In some embodiments, the drug delivery systems can include a label associated with a housing, such as housing 30 shown in FIG. 5B, and/or a drug holder of the drug administration device. The label can be configured to provide visual indication to a user that the drug disposed within the drug holder has exceeded a predetermined exposure threshold for the drug. The predetermined exposure threshold can be associated with an exposure condition of the drug, e.g., temperature, UV exposure, etc.

For example, the predetermined exposure threshold can be a temperature threshold. The temperature threshold can include at least one of an absolute minimum temperature threshold, an absolute maximum temperature threshold, and a duration threshold below the absolute minimum temperature threshold or above the absolute maximum temperature threshold. In certain instances, a temperature threshold may be desired, particularly in instances where the drug is sensitive to temperature changes. Examples of temperature sensitive drugs include golimumab, ustekinumab, daratumumab, esketamine, ketamine, and guselkumab.

The label can include a variety of materials. In some embodiments, the label can include at least one electrochromic material and/or at least one thermochromic material. Examples of suitable thermochromic materials include at least one thermochromic ink. Thermochromic ink is configured to change color in response to temperature. Thermochromic ink has been used in consumer beverage packaging to show if the product is warm or cold and can be sunlight activated. Thermochromic ink has also been used in some forms to create glow-in-the-dark inks. Alternatively, or in addition, the label can include a reactive agent that is configured to interact with the drug within the drug holder so as to trigger a visual change of at least a portion of the label when the drug has exceeded a predetermined exposure threshold.

In one embodiment, the label includes at least one electrochromic material, e.g., electrochromic ink. Non-limiting examples of suitable electrochromic materials include at least one electrochromic ink. Electrochromic ink is configured to change color when an electric current is applied thereto. Electrochromic inks have been used in voltage checks on batteries and can be used within electrical circuits to indicate when a button, circuit, or portion the system is active. The electrochromic material can be configured to be a first color while in a first state, and then when transitioned to a second state, visually present a second color that is different than the first color. Alternatively, the electrochromic material can be configured to change its transparency state. In this way, the electrochromic material can be placed over printed information that would not be visible while the indicator is in a first state, but then when transitioned, information below could be viewed through the now transparent electrochromic material. Further, while the electrochromic material is described as having two states, a person skilled in the art will appreciate that some electrochromic materials can have more than two stable states. In use, the electrochromic material can be transitioned according to defined criteria stored within a processor for the particular drug associated with the label. As such, when the defined criteria has been satisfied, the processor transmits an electronic signal to the label to cause the electrochromic material to transition from the first state to the second state. For example, the defined criteria can be a temperature threshold for the drug.

The visual indication can be in a variety of forms, for example one or more words, numbers, letters, shapes, symbols, continuous or discontinuous designs or patterns, or any combination thereof. Alternatively, or in addition, the visual indication is a color change of at least a portion of the label from a first color to a second color that is different than the first color.

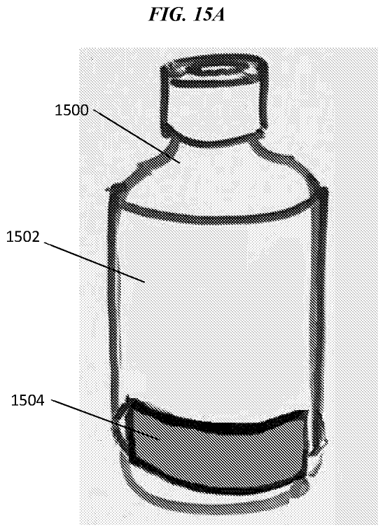

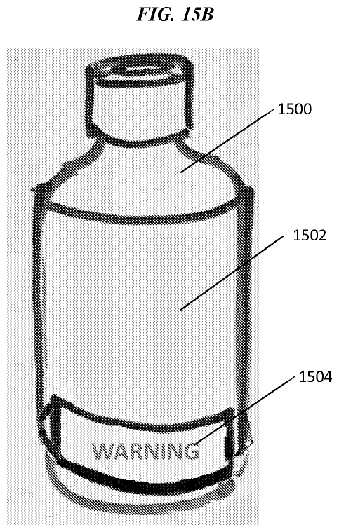

FIGS. 15A and 15B illustrate an exemplary drug holder 1500 having a label 1502 disposed thereon. While not shown, a drug is disposed within the drug holder 1500. In this illustrated embodiment, the label 1502 includes an electrochromic ink 1504 printed thereon. The electrochromic ink 1504 is configured to provide a visual indication in response to the drug exceeding a temperature threshold. FIG. 15A shows the label 1502 in a first state in which the drug has not exceeded the temperature threshold. As shown, when the label 1502 is in the first state, the electrochromic ink 1504 is in a static initial state. That is, the electrochromic ink 1504 has not been triggered by a processor (not shown), which is in communication therewith, to transition to another state. In contrast, FIG. 15B shows the label 1502 in a second state in which the drug has exceeded the temperature threshold. As shown, when the label 1502 is in the second state, the electrochromic ink 1504 has transitioned from its initial state in such a way that formed the word “WARNING.”

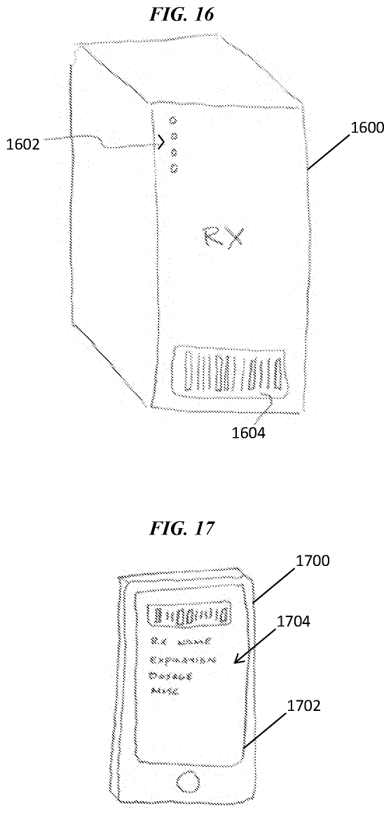

FIG. 16 illustrates an exemplary housing 1600, e.g., package, for a drug administration device (not shown) having a drug disposed within a drug holder thereof. The housing 1600 has color-changing material 1602 thereon. The color-changing material 1602 in this illustrated embodiment includes four dots printed with electrochromic ink, although another number of dots may be used and/or another color-changing material may be used. As discussed herein, the color-changing material 1602 is configured to provide a visual indication in response to the drug exceeding a temperature threshold. FIG. 16 shows the color-changing material 1602 in a first state in which the drug has not exceeded the temperature threshold, similar to the first state of the label 1502 of FIG. 15A. The color-changing material 1602 is configured to change to a second state in response to exceeding a temperature threshold, similar to the second state of the label 1502 of FIG. 15B. In this illustrated embodiment, the first state of the color-changing material 1602 is a first color, and the second state of the color-changing material 1602 is a second, different color.

The housing 1600 in the embodiment of FIG. 16 also has a label 1604 thereon. In other embodiments, the housing 1600 can include the label 1604 but not the color-changing material 1602 or can include the color-changing material 1602 but not the label 1604. The label 1604 is configured to be electrically connected to a sensor (not shown), similar to that discussed above. The sensor is disposed within the housing 1600 and is associated with the drug administration device disposed in the housing 1600 or with the drug holder of the drug administration device disposed in the housing 1600.

The label 1604 is configured to be scanned by a reader to provide data monitored by the sensor, e.g., any one or more of temperature data, UV data, pH data, pressure data, etc., and thereby provide exposure condition information of the drug. The label 1604 can be scanned by any of a variety of readers configured to read data for the particular type of label 1604. For example, the label 1604 can include a radio frequency identification (RFID) tag, and the reader can include an RFID reader. For another example, the label 1604 can include a barcode (as in this illustrated embodiment), and the reader can be a device configured to read a barcode. FIG. 17 illustrates an exemplary reader 1700 configured to read the label 1604. The reader 1700 is a smartphone in this illustrated embodiment, but, as will be appreciated by a person skilled in the art, other types of devices can be configured to read a barcode. The reader 1700 can be configured to provide information regarding the scanned data to a user of the reader 1700. In this illustrated embodiment, the reader 1700 is configured to show information 1702 regarding the scanned data on a display screen 1704 of the reader 1700. The information 1702 regarding the scanned data in this illustrated embodiment includes an image of the barcode on the label 1604 and information related to the drug administration device and the drug, including a name of the drug, an expiration date of the drug, a dosage of the drug as prescribed, and miscellaneous information. The expiration date of the drug can be determined by a processor of the reader 1700 as discussed herein using data sensed by the sensor disposed within the housing 1600 and communicated to the reader 1700 via scanning of the label 1604. The miscellaneous information can include any of a variety of types of information, such as a name of the patient prescribed the drug, a name of the patient's prescribing physician, contact information for the patient's prescribing physician, contact information for a pharmacy that provided the housing 1600, a message indicating that the drug has not exceeded a temperature threshold (and/or other threshold as appropriate for the type of condition sensed) and is therefore in a safe condition for use, a message indicating that the patient should wait an amount of time before room temperature may bring the drug to a more comfortable temperature for drug delivery given the sensed current temperature, a warning message if the drug has exceeded a temperature threshold (and/or other threshold as appropriate for the type of condition sensed) and is thus no longer safe for use, etc.

In some embodiments of a passive visual confirmation system, a color can indicate whether or not a housing, e.g., package, for a drug administration device has been opened, which can be an indication of tampering or premature handling if the color is present before intended or expected by a user. The housing can be configured to change color in response to being exposed to ambient air. In other words, the housing can be a first color while in a first state, which corresponds to a sealed or closed housing, and can be configured to transition to a second color in a second state, which corresponds to the housing being at least partially open or having been previously at least partially opened. In an exemplary embodiment, the housing can be configured to change color when exposed to oxygen, e.g., to change from the first state to the second state when exposed to oxygen. The housing can be charged with an element other than oxygen, such as nitrogen, such that the color changes automatically when the housing is at least partially opened to allow entry of oxygen into the housing. The housing can have a variety of configurations, such as a blister pack in which the drug administration device is contained with the blister charged with the element other than oxygen. In this way, when the drug administration device is released from the blister, the housing automatically changes color so as to indicate release of the blister.

In some embodiments of a passive visual confirmation system, a color can indicate whether or not a drug has been rendered non-viable for use. A bio-safe reactive agent can be mixed with the drug. The reactive agent can be configured to change color if exposed to an environmental condition, such as ambient air or a threshold temperature, that renders the drug non-viable for use. The changed color can thus be indicative that the drug has been exposed to the environmental condition and has thus been rendered non-viable for use.

The present disclosure has been described above by way of example only within the context of the overall disclosure provided herein. It will be appreciated that modifications within the spirit and scope of the claims may be made without departing from the overall scope of the present disclosure.

What is claimed is:

1. A drug delivery system, comprising:
a drug administration device including a drug holder having a drug disposed therein, the drug administration device being configured to deliver the drug;
a first sensor associated with at least one of the drug administration device and a packaging unit for the drug administration device, the first sensor being configured to monitor at least one exposure condition of the drug after being associated with the drug administration device; and
a second sensor associated with the drug and configured to monitor at least one exposure condition of the drug from an initial time before the drug is associated with the drug administration device to a second time in which the drug is associated with the drug administration device and the first sensor is activated.

2. The system of claim 1, wherein the drug administration device further includes a drug dispensing mechanism configured to deliver at least a portion of the drug upon actuation of a drug delivery actuator by a user.

3. The system of claim 1, wherein the drug administration device is one of a blister pack, an autoinjector, an infusion pump, and an inhaler.

4. The system of claim 1, further comprising a communications interface configured to communicate with a processor.

5. The system of claim 4, wherein the processor is one of a processor remote from the drug administration device and a processor local to the drug administration device.

6. The system of claim 1, wherein the at least one exposure condition is at least one of geographic location, time, date, temperature, UV exposure, and humidity.

7. The system of claim 1, wherein the first sensor is on the packaging unit and the packaging unit contains one or more drug administration devices.

8. The system of claim 1, wherein the initial time is the time that the drug enters the supply chain.

9. The system of claim 4, wherein data collected by at least one of the first sensor and the second sensor is configured to be communicated to the processor via the communications interface.

10. The system of claim 9, wherein the processor is configured to determine an expiration date for at least one of a batch of the drug and the drug in the drug administration device.

11. The system of claim 10, wherein the processor is configured to generate a warning to a user in response to determining that at least one of a batch of the drug is beyond its expiration date and the drug in the drug administration device is beyond its expiration date.

12. The system of claim 10, wherein the drug administration device is configured to prevent drug delivery in the event that at least one of a batch of the drug is beyond its expiration date and the drug in the drug administration device is beyond its expiration date.

13. The system of claim 1, wherein the drug comprises at least one of infliximab, golimumab, ustekinumab, daratumumab, guselkumab, epoetin alfa, risperidone, and paliperidone palmitate.

14. A method, comprising:
monitoring, by a first sensor, at least one exposure condition of a drug after the drug is associated with a drug administration device;
transmitting data representative of the at least one exposure condition to a communications interface in communication with the first sensor;
receiving and transmitting, by the communications interface, the data to a processor that is in communication with the communications interface; and
determining, by the processor, viability of the drug based on the received data characterizing the at least one exposure condition.

15. The method of claim 14, further comprising
monitoring, by a second sensor, at least one exposure condition of the drug during a time interval that is at least prior to the drug being associated with the drug administration device;
transmitting data representative of the at least one exposure condition over the time interval to the communications interface in communication with the second sensor;
receiving and transmitting, by the communications interface, the data to the processor; and
determining, by the processor, viability of the drug based on the received data characterizing the at least one exposure condition.

16. The method of claim 15, wherein the time interval includes when the drug enters the supply chain.

17. The method of claim 14, wherein the drug comprises at least one of infliximab, golimumab, ustekinumab, daratumumab, guselkumab, epoetin alfa, risperidone, and paliperidone palmitate.

18. A drug delivery system, comprising:
a housing containing one or more drug administration devices, each drug administration device including at least one drug holder having a drug disposed therein, and further being configured to deliver the drug;
a sensor associated with at least one of the housing, the one or more drug administration devices, and the at least one drug holder, the sensor being configured to detect at least one exposure condition of the drug; and
a communications interface configured to communicate the at least one exposure condition of the drug to a processor.

19. The system of claim 18, wherein the housing is a packaging unit for the one or more drug administration devices.

20. The system of claim 18, wherein at least one of the one or more drug administration devices further includes a drug dispensing mechanism configured to deliver the drug upon actuation of a drug delivery actuator by a user.

21. The system of claim 18, wherein at least one of the one or more drug administration device is one of a blister pack, an autoinjector, an infusion pump, and an inhaler.

22. The system of claim 18, wherein the processor is one of a processor remote from the one or more drug administration devices and a processor local to the one or more drug administration devices.

23. The system of claim 22, wherein the at least one exposure condition is at least one of geographic location, time, date, temperature, UV exposure, and humidity.

24. The system of claim 23, wherein the communications interface is configured to communicate data representative of the exposure condition to the processor at one of a regular sampling rate, on demand, and continuously.

25. The system of claim 18, wherein the sensor is configured to sense at least one of an intensity of the at least one exposure condition and a duration of the at least one exposure condition.

26. The system of claim 18, wherein at least one of the one or more drug administration devices further comprises a local processor configured to at least one of adjust a dosage of the drug based on the at least one exposure condition of the drug and adjust a rate of delivery of the drug from the one or more drug administration devices based on the at least one exposure condition of the drug.

27. The system of claim 18, further comprising an indicator associated with at least one of the at least one drug holder and the one or more drug administration devices that is configured to communicate a condition of the drug to a user.

28. The system of claim 27, wherein the condition of the drug is at least one of an expiration date of the drug, a viability state of the drug, a potency of the drug, and a recommended dosage of the drug.

29. The system of claim 18, wherein the drug comprises at least one of infliximab, golimumab, ustekinumab, daratumumab, guselkumab, epoetin alfa, risperidone, and paliperidone palmitate.

30. A method, comprising:
detecting, by at least one sensor, at least one exposure condition of a drug;
transmitting data representative of the at least one exposure condition to a communications interface in communication with the at least one sensor;
receiving and transmitting, by the communications interface, the data to a processor that is in communication with the communications interface; and
determining, by the processor, viability of the drug based on the received data characterizing the at least one exposure condition.

31. The method of claim 30, wherein the drug comprises at least one of infliximab, golimumab, ustekinumab, daratumumab, guselkumab, epoetin alfa, risperidone, and paliperidone palmitate.

32. A drug delivery system, comprising:
a drug administration device including a drug holder having a drug disposed therein, the drug administration device being configured to deliver the drug; and
a drug status indicator associated with at least one of the drug administration device and the drug holder, the drug status indicator being configured to indicate an extent of an exposure of the drug to an environmental condition; and
a reader associated with the drug administration device, the reader being configured to detect the drug status indicator.

33. The system of claim 32, wherein the drug status indicator is on a housing for at least one of the drug administration device and the drug holder.

34. The system of claim 33, wherein the housing is a packaging unit for one or more drug administration devices.

35. The system of claim 32, wherein the drug administration device further includes a drug dispensing mechanism configured to deliver the drug upon actuation of a drug delivery actuator by a user.

36. The system of claim 32, wherein the drug administration device is one of a blister pack, an autoinjector, an infusion pump, a nasal spray device, and an inhaler.

37. The system of claim 32, wherein the environmental condition is at least one of temperature, UV exposure, pH, and humidity.

38. The system of claim 32, wherein the drug status indicator is configured to be responsive to at least one of an intensity of the environmental condition and a duration of the environmental condition.

39. The system of claim 32, wherein the drug status indicator is a degradable element that is configured to degrade in response to at least one of a threshold exposure duration and a threshold exposure intensity to the environmental condition, and wherein the reader is configured to be in communication with a processor that is configured to prompt a cue to a user when at least one of the threshold exposure duration and the threshold exposure intensity has been exceeded.

40. The system of claim 39, wherein the cue is at least one of an audible cue and a visual cue.

41. The system of claim 32, wherein the drug status indicator is configured to undergo a color change in response to at least one of a threshold exposure duration or a threshold exposure intensity to the environmental condition, and wherein the reader is configured to be in communication with a processor that is configured to prompt a cue to a user when at least one of the threshold exposure duration and the threshold exposure intensity has been exceeded.

42. The system of claim 32, wherein the drug status indicator includes a reactive agent that is configured to interact with the drug so as to provide a visual change that is configured to be detected by the reader when the drug is below at least one of a threshold exposure duration and a threshold exposure intensity to the environmental condition.

43. The system of claim 32, wherein the drug status indicator is formed of at least one electrochromic material.

44. The system of claim 32, wherein the drug status indicator is formed of at least one of polylactic acid, polyglycolic acid, polycaprolactone, and polydioxanone.

45. The system of claim 32, wherein the drug holder includes a first vial configured to have a lyophilized component of the drug disposed therein and a second vial configured to have a diluent disposed therein, the lyophilized component is configured to be mixed with the diluent to reconstitute the drug prior to delivery of the drug, and the drug status indicator integrated with the drug holder and is configured to indicate whether the lyophilized component and the diluent are each in a safe state for mixing.

46. The system of claim 32, wherein the drug holder includes a first vial configured to have a lyophilized component of the drug disposed therein and a second vial configured to have a diluent disposed therein, the lyophilized component is configured to be mixed with the diluent to reconstitute the drug prior to delivery of the drug, and the drug status indicator is configured to be releasably and replaceably attached to the drug administration device and is configured to indicate whether the lyophilized component and the diluent are each in a safe state for mixing.

47. The system of claim 32, wherein the drug comprises at least one of infliximab, golimumab, ustekinumab, daratumumab, guselkumab, epoetin alfa, risperidone, esketamine, ketamine, and paliperidone palmitate.

48. A drug delivery system, comprising:
a drug administration device including a housing and a drug holder having a drug disposed therein, the drug administration device being configured to deliver the drug; and
a label associated with at least one of the housing and the drug holder that is configured to provide a visual indication to a user that the drug has exceeded a temperature threshold for the drug.

49. The system of claim 48, wherein the temperature threshold includes at least one of an absolute minimum temperature threshold, an absolute maximum temperature threshold, and a duration threshold below the absolute minimum temperature threshold or above the absolute maximum temperature threshold.

50. The system of claim 48, wherein the visual indication is a color change of at least a portion of the label from a first color to a second color that is different than the first color.

51. The system of claim 48, wherein the label includes at least one of a thermochromic material and an electrochemical material.

52. The system of claim 51, wherein the drug comprises at least one of infliximab, golimumab, ustekinumab, daratumumab, esketamine, ketamine, and guselkumab.

53. The system of claim 48, wherein the label includes a reactive agent that is configured to interact with the drug so as to trigger a visual change of at least a portion of the label when the drug has exceeded a temperature threshold.

54. A method, comprising:
monitoring, by a drug status indicator associated with at least one of a drug administration device and a drug holder having a drug disposed therein, an extent of an exposure of the drug to at least one environmental condition;
sensing, by a reader associated with the drug administration device, the drug status indicator to detect a response that is indicative of the extent of the exposure of the drug to the at least one environmental condition;
transmitting data representative of the response of the drug status indicator to a processor in communication with the reader; and
determining, by the processor, viability of the drug based on the received data characterizing the response of the drug status indicator.

55. The method of claim 54, wherein the drug comprises at least one of infliximab, golimumab, ustekinumab, daratumumab, guselkumab, epoetin alfa, risperidone, esketamine, ketamine, and paliperidone palmitate.
